(19) United States
(12) Patent Application Publication
(10) Pub. No.: US 2025/0306040 A1
(43) Pub. Date: Oct. 2, 2025

(54) METHODS AND KITS FOR DETECTION OF AMYLIN-BETA AMYLOID CO-AGGREGATION

(71) Applicant: University of Kentucky Research Foundation, Lexington, KY (US)

(72) Inventors: Florin Despa, Lexington, KY (US); Deepak Kotiya, Lexington, KY (US); Gregory A. Jicha, Lexington, KY (US); Larry B. Goldstein, Lexington, KY (US)

(21) Appl. No.: 19/096,342

(22) Filed: Mar. 31, 2025

Related U.S. Application Data
(60) Provisional application No. 63/573,040, filed on Apr. 2, 2024.

(51) Int. Cl. G01N 33/74; G01N 33/68

(52) U.S. Cl. CPC G01N 33/74; G01N 33/6893; G01N 33/6896; G01N 2800/042; G01N 2800/2821

(57) ABSTRACT

Methods for detecting or quantifying amylin-beta amyloid (Aβ) hetero-oligomers (amylin-Aβ aggregate) are provided. Anti-amylin and anti-Aβ antibodies which recognize epitopes that are distinct from high affinity binding sites between amylin peptide and Aβ peptide can be utilized as capture and detection antibodies, respectively, in a sandwich enzyme-linked immunosorbent assay (ELISA) to provide detection and quantification of amylin-Aβ aggregate present in a biological sample, such as blood or brain tissue. Kits useful for the detection and the quantification of amylin-Aβ aggregate are also provided.

### CROSS-REFERENCE TO RELATED APPLICATION

The present application claims priority to U.S. Patent Application Ser. No. 63/573,040 filed on Apr. 2, 2024, the entire disclosure of which is incorporated herein by reference.

### GOVERNMENT INTEREST

This invention was made with government support under grant number R01 NS116058, R01 AG057290, and R01 AG053999, awarded by the National Institutes of Health. The government has certain rights in the invention.

### REFERENCE TO AN ELECTRONIC SEQUENCE LISTING

The contents of the electronic sequence listing (UKRF 2775US Sequence Listing.xml; Size: 8,966 bytes; and Date of Creation: Mar. 29, 2025) is herein incorporated by reference in its entirety.

### TECHNICAL FIELD

The presently disclosed subject matter generally relates to the detection of amylin-beta amyloid (Aβ) hetero-oligomers (amylin-Aβ aggregate). In particular, certain embodiments of the presently disclosed subject matter relate to methods and kits which utilize an anti-AB antibody and an anti-amylin antibody designed to recognize epitopes which are distinct from high affinity binding sites between amylin peptide and Aβ peptide to detect and quantify amylin-Aβ aggregate.

### BACKGROUND

Amylin, also referred to as islet amyloid polypeptide (IAPP), secreted from the pancreas crosses from the blood to the brain parenchyma and forms cerebral mixed amylin-beta amyloid (Aβ) plaques, which are found in both sporadic and early-onset familial Alzheimer's disease (AD). However, the role of amylin-Aβ co-aggregation as the potential mechanism underlying this association remains unknown, in part due to lack of assays for detection of these complexes. In this regard, while the need to quantify molecular amylin-Aβ interaction in human Alzheimer's disease (AD) has been recognized, the complexity and lack of scalability of traditional methods (such as immunoprecipitation, Western blot, circular dichroism, and electron microscopy) have hampered studies requiring larger sample sizes.

### SUMMARY

The presently disclosed subject matter meets some or all of the above-identified needs, as will become evident to those of ordinary skill in the art after a study of information provided in this document.

This summary describes several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this summary does not list or suggest all possible combinations of such features.

The presently disclosed subject matter, in one aspect, includes methods for detecting or quantifying amylin-beta amyloid (Aβ) aggregate. In some embodiments, a method to detect or quantify amylin-Aβ aggregate involves: contacting a capture antibody that specifically binds to one or more epitopes of amylin-Aβ aggregate with a biological sample having amylin-AB aggregate present therein to form an initial (or “first”) complex; and contacting a detection antibody with the amylin-AB aggregate present in the first complex to form a subsequent (or “second”) complex between the capture antibody, the amylin-Aβ aggregate, and detection antibody. A detection enzyme may be conjugated (linked) to the detection antibody to facilitate the detection and/or quantification of the amylin-AB aggregate from the biological sample. In some embodiments, the method further involves contacting the detection enzyme linked to the detection antibody with a substrate and detecting and/or quantifying the amylin-Aβ aggregate from the biological sample using the signal produced by the reaction between the substrate and the detection enzyme. In some embodiments, the method further involves coating a solid surface with the capture antibody to immobilize amylin-Aβ aggregate present in the biological sample upon contacting the capture antibody. As such, in some embodiments, contacting the capture antibody and the biological sample may involve contacting the coated solid surface with the biological sample.

In some embodiments, the capture antibody is an anti-amylin antibody and the detection antibody is an anti-AB antibody. In some embodiments, the capture antibody is a polyclonal antibody and the detection antibody is a monoclonal antibody. In some embodiments, the anti-amylin antibody specifically binds to at least two epitopes, each comprising a sequence selected from SEQ ID NO: 3, SEQ ID NO: 4, and SEQ ID NO: 5. In some embodiments, the anti-amylin antibody specifically binds to a first epitope comprising SEQ ID NO: 3, a second epitope comprising SEQ ID NO: 4, and a third epitope comprising SEQ ID NO: 5. In some embodiments, the epitope that the anti-AB antibody specifically binds to comprises SEQ ID NO: 6. In various embodiments, some or all of the epitopes to which the anti-amylin antibody specifically binds is an exposed epitope of amylin peptide of amylin-Aβ aggregate. In some embodiments, the epitope to which the anti-Aβ antibody specifically binds is an exposed epitope of an Aβ peptide of an amylin-Aβ aggregate.

In some embodiments, the capture antibody includes one or more paratopes comprising a sequence having homology to the amino acid sequence of SEQ ID NO: 7. In some embodiments, paratope(s) of the capture antibody may comprise a sequence which is at least 90% homologous to the amino acid sequence of SEQ ID NO: 7. In some embodiments, the detection antibody comprises a sequence selected from SEQ ID NO: 8, SEQ ID NO: 9, and SEQ ID NO: 10.

In some embodiments, the biological sample is a blood sample. In some embodiments, the biological sample is brain tissue. In some embodiments, the biological sample is obtained from a subject suffering from type-2 diabetes. In some embodiments, the biological sample is obtained from subject a suffering from Alzheimer's disease. In some embodiments, the amlylin-Aβ aggregate from the biological sample is prefibrillar amylin-Aβ aggregate.

The presently disclosed subject matter, in another aspect, includes a kit useful for amylin-Aβ aggregate detection or quantification. In some embodiments, a kit for amylin-AB aggregate detection or quantification includes: an anti-amylin antibody that specifically binds to one or more epitopes of amylin-AB aggregate; and an anti-Aβ antibody that specifically binds to an epitope of the amylin-AB aggregate that is distinct from the one or more epitopes of the amylin-Aβ aggregate that the anti-amylin antibody specifically binds to.

In some embodiments, the one or more epitopes that the anti-amylin antibody of the kit specifically binds to includes an epitope comprising sequence selected from SEQ ID NO: 3, SEQ ID NO: 4, and SEQ ID NO: 5. In some embodiments, the anti-amylin antibody is a polyclonal antibody. In some embodiments, the anti-amylin antibody of the kit specifically binds to at least two epitopes, each comprising a sequence selected from SEQ ID NO: 3, SEQ ID NO: 4, and SEQ ID NO: 5. In some embodiments, the anti-amylin antibody specifically binds to a first epitope comprising SEQ ID NO: 3, a second epitope comprising SEQ ID NO: 4, and a third epitope comprising SEQ ID NO: 5. In some embodiments, the anti-AB antibody of the kit is a monoclonal antibody. In some embodiments, the epitope that the anti-AB antibody specifically binds to comprises SEQ ID NO: 6. In various embodiments, some or all of the epitopes to which the anti-amylin antibody of the kit specifically binds is an exposed epitope of amylin peptide of amylin-AB aggregate. In some embodiments, the epitope to which the anti-AB antibody of the kit specifically binds is an exposed epitope of an Aβ peptide of an amylin-Aβ aggregate.

In some embodiments, the anti-amylin antibody of the kit includes one or more paratopes comprising a sequence having homology to the amino acid sequence of SEQ ID NO: 7. In some embodiments, paratope(s) of the anti-amylin antibody may comprise a sequence which is at least 90% homologous to the amino acid sequence of SEQ ID NO: 7. In some embodiments, the anti-AB antibody of the kit comprises a sequence selected from SEQ ID NO: 8, SEQ ID NO: 9, and SEQ ID NO: 10.

In some embodiments, the kit further includes a detection enzyme linked to the anti-Aβ antibody. In some embodiments, the kit further includes a microplate including a well for receiving the anti-amylin antibody and the anti-AB antibody. In some embodiments, the kit further includes a washing buffer. In some embodiments, the kit further includes a substrate for reaction with the detection enzyme. In some embodiments, the kit further includes a stop solution for terminating a reaction between the substrate and the detection enzyme.

In some embodiments, the kit includes: a polyclonal anti-amylin antibody that specifically binds to two or more epitopes of amylin-AB aggregate, with each epitope of the two or more epitopes comprising a sequence selected from SEQ ID NO: 3, SEQ ID NO: 4, and SEQ ID NO: 5; and a monoclonal anti-AB antibody that specifically binds to an epitope of the amylin-Aβ aggregate comprising SEQ ID NO: 6. In one such embodiment, the anti-amylin antibody includes multiple paratopes, with each paratope of the multiple paratopes comprising a sequence which is at least 90% homologous to the amino acid sequence of SEQ ID NO: 7, and the anti-AB antibody includes a paratope comprising SEQ ID NO: 8.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIGS. 1A-1F. Detection of amylin-AB hetero-oligomers in human AD brain tissues. FIG. 1A is a schematic illustration of amylin-Aβ hetero-amyloid formation in the brain along with an example of cerebral amylin-AB co-deposition previously reported. The section through the brain of a presenlin 1 (PS1) mutation carrier shows cerebral Aβ deposits (green) and amylin (brown) forming the plaque core and intercalated amylin-Aβ deposits. FIG. 1B is a confocal microscopic analysis of a section through the brain of PS1 mutation carrier stained with Thioflavin S (Thio-S) (green), amylin (red), AB (magenta), and overlay (orange). Scale bar: 10 μm. FIG. 1C is a confocal microscopic analysis of a section through the brain of a person with SAD stained with amylin (green) and AB (red), and further subjected to an amylin-Aβ proximity ligation assay (PLA) (red). Scale bar: 20 μm. FIG. 1D is a schematic representation of an amylin-Aβ sandwich ELISA with the anti-amylin antibody used as the capture antibody and anti-Aβ antibody conjugated with horse radish peroxidase (HRP) used as the detection antibody. FIG. 1E is an amino acid sequence analysis of amylin and Aβ peptides indicating the epitope regions against the anti-amylin capture and anti-Aβ detection antibodies used in the amylin-Aβ sandwich ELISA. FIG. 1F are charts showing amylin-Aβ immuno-reactivity signal intensity measured in human sAD brain tissue homogenates (n=5) using the P2 amylin antibody as the capture antibody, and total Aβ or Aβ₄₂ or Aβ₄₀ antibodies as the detection antibody in the amylin-Aβ sandwich ELISA.

FIGS. 2A-2H. Specificity and sensitivity of the amylin-Aβ sandwich ELISA to detection of amylin-Aβ hetero-oligomers. FIG. 2A is a chart showing average amylin-Aβ immuno-reactivity signal intensities measured in amylin-Aβ (30 μM amylin and 16.2 μM Aβ; mixed in a 1:1 ratio to give final concentrations of 15 μM amylin and 8.1 μM Aβ), amylin (30 μM), and Aβ (16.2 μM) aggregates using the amylin-Aβ sandwich ELISA. FIG. 2B is a chart showing Thioflavin T (Th-T) fluorescence signal intensities measured in solutions of human amylin-Aβ and rat amylin-Aβ aggregates (same peptide concentrations as in FIG. 2A). FIG. 2C is a chart showing amylin-Aβ immuno-reactivity signal intensities measured by the amylin-Aβ sandwich ELISA in rat amylin-Aβ and human amylin-Aβ hetero-oligomers. FIG. 2D is a chart showing amylin-Aβ immuno-reactivity signal intensities measured by the amylin-Aβ sandwich ELISA in amylin-Aβ dilutions (1:10 to 1:160) using the same aliquots of amylin-Aβ as in FIG. 2B. FIG. 2E shows co-immunoprecipitation of amylin using the P2 amylin antibody in amylin-Aβ aggregates (undiluted and 1:64 diluted) followed by Western blot analysis of Aβ and amylin in amylin IP eluates. FIG. 2F is a chart showing amylin-Aβ immuno-reactivity signal intensities measured by the amylin-Aβ sandwich ELISA in the fraction collected at various time points of human amylin (30 μM) incubated with human Aβ₄₀ (16.2 μM). FIG. 2G is a chart showing Thioflavin T (Th-T) fluorescence signal intensities measured in the same fraction collected as in FIG. 2F. FIG. 2H is a chart showing pairwise correlation analyses of amylin-Aβ signal intensity measured in FIG. 2F versus. Th-T fluorescence signal intensities measured in FIG. 2G. Data are mean±S.D. Data are presented as correlation analysis, Pearson's correlation **P<0.01 in FIG. 2H.

FIGS. 3A-3E. Sensitivity of amylin-Aβ sandwich ELISA to dissociation of amylin-Aβ hetero-oligomers. FIG. 3A is a schematic illustration describing APP/PS1/HIP rats expressing human amylin in the pancreas. (APP=amyloid precursor protein). FIG. 3B is a chart showing amylin-Aβ concentrations measured using the amylin-Aβ sandwich ELISA in brain homogenates from age-matched APP/PS1 and APP/PS1/HIP rats (age, 16-months, n=7 males/group) with and without hydrochloric acid (HCl) treatment to fragment the amylin-Aβ hetero-oligomers. FIG. 3C is a chart showing pairwise comparison for the level of amylin-Aβ concentrations measured in FIG. 3B with and without hydrochloric acid (HCl) treatment for APP/PS1/HIP brain homogenates. FIG. 3D is a chart showing amylin concentrations measured in same samples as in FIG. 3B using indirect amylin ELISA. FIG. 3E is a chart showing pairwise comparison of the levels of amylin concentrations measured in FIG. 3D with and without hydrochloric acid (HCl) treatment for APP/PS1/HIP brain homogenates. Data are mean±S.D. Kruskal-Wallis one-way of variance test in FIG. 3B *P<0.05 and FIG. 3D. Pairwise comparisons and estimation plots, two-tailed, paired t-test *P<0.05 in FIG. 3C and FIG. 3E.

FIGS. 4A-4F. Correlations between brain tissue amylin-Aβ, amylin, and Aβ concentrations with CERAD score of sAD brains. FIGS. 4A-4C show brain tissue amylin-Aβ concentrations (FIG. 4A) measured using the amylin-Aβ sandwich ELISA in APP and PS1 mutation carriers (fAD; n=18), persons with sAD (n=45), and non-AD individuals (n=13)) along with Aβ concentrations measured by total Aβ ELISA (FIG. 4B) and amylin concentrations measured by amylin ELISA (FIG. 4C) in the same brain tissue homogenates as in FIG. 4A. FIGS. 4D-4F show the pairwise correlation analysis of CERAD scores of sAD and non-AD brains versus amylin-Aβ concentrations (FIG. 4D), CERAD scores of sAD and non-AD brains versus brain tissue amylin concentrations (FIG. 4E), and CERAD scores of sAD and non-AD brains versus brain tissue Aβ concentrations (FIG. 4F). Number distribution of brains stratified by the CERAD score included CERAD none (n=12), CERAD A (n=6), CERAD B (n=15) and CERAD C (n=22). n=3 sAD brains that have CERAD none and Braak scores III-IV were excluded from the analyses (FIGS. 4D-4F). Data are mean #S.D. One-way ANOVA test in (FIGS. 4A-4C) **P<0.01, ***P<0.001 and ****P<0.0001. Data are presented as correlation analysis, Pearson's correlation in FIGS. 4D-4F, *P<0.05.

FIGS. 5A-5C. The relationships between amylin-Aβ oligomerization and brain amylin and Aβ levels in age-related AD. FIG. 5A is a chart showing pairwise correlation analyses of brain tissue amylin-Aβ concentrations versus age for sAD and non-AD groups. FIG. 5B is a chart showing brain amylin-Aβ hetero-oligomer levels as a function of brain amylin levels. Regression line and confidence interval for the mean are shown for subjects in the AD group (n=46). The P value shown was calculated by linear regression for the effect of brain amylin levels on the amylin-Aβ hetero-oligomerization with adjustment for brain Aβ level (P=0.05) and a multiplicative (amylin×Aβ) interaction term (P=0.13, after including the main effect terms (brain amylin and Aβ levels). FIG. 5C is a chart showing the same as in above for the non-AD group (n=13).

FIGS. 6A-6E. The relationships between amylin-Aβ oligomerization and brain amylin and Aβ levels in the setting of type-2 diabetes. FIG. 6A is a chart showing brain tissue amylin-Aβ concentrations measured using the amylin-Aβ sandwich ELISA in groups of individuals stratified by type-2 diabetes mellitus (diabetes group n=28; non-diabetes group; n=30). FIG. 6B is a chart showing brain amylin-Aβ hetero-oligomer levels as a function of brain amylin levels. Regression line and confidence interval for the mean are shown for subjects in the diabetes group. The P value shown was calculated by linear regression for the effect of brain amylin level on the amylin-Aβ hetero-oligomerization with adjustment for brain Aβ level (P<0.05) and a multiplicative (amylin×Aβ) interaction term (P<0.05). FIG. 6C is a chart showing a Goodness-of-Fit test for a multiple logistic regression model based on brain amylin, amylin-Aβ and age as covariates and a logistic model based on brain Aβ level and age as predictor variables. Receiver Operating Characteristic (ROC) curves were created plotting sensitivity versus specificity for all possible cut points from fitted values in a model based on brain amylin, amylin-Aβ and age (pink curve) versus a model based on brain Aβ level and age (gray curve). FIG. 6D is a chart showing the same as in FIG. 6B but for the non-diabetes group. FIG. 6E is a chart showing the same as in FIG. 6C but for the non-diabetes group.

FIGS. 7A-7F. Detection of amylin-Aβ hetero-oligomers in the blood sampled from APP/PS1/HIP and APP/PS1 rats. FIG. 7A is an illustration showing differences in the amino acid sequences of human versus rat amylin peptides. FIGS. 7B-7C are charts showing amylin-Aβ hetero-oligomer concentrations measured in whole blood (FIG. 7B) and plasma (FIG. 7C) from age-matched APP/PS1/HIP and APP/PS1 rats (age, 16-months, n=10 males/group) by using the amylin-Aβ sandwich ELISA. FIGS. 7D-7E are representative images of confocal microscopic analysis of red blood cells from APP/PS1/HIP rats (n=5) triple-stained with anti-amylin antibody (green), anti-Aβ antibody (magenta), and hemoglobin (red) (FIG. 7D), as well as triple-stained with anti-amylin antibody (green), anti-Aβ antibody (magenta), and glycophorin (red) (FIG. 7E). FIG. 7F is a schematic summary of the results: amyloid-forming human amylin secreted from the pancreas into the blood forms mixed amylin-Aβ hetero-oligomers that accumulates in circulating RBCs and within the brain that can be detected by the amylin-Aβ sandwich ELISA. Data are means±S.D., unpaired two-tailed t-test *P<0.05 in FIG. 7B; Scale Bars 2 μm in FIG. 7D and 2 μm in FIG. 7E.

FIG. 8 is a chart showing comparative analyses of amylin-Aβ immuno-reactivity signal intensities measured in amylin-Aβ solution phase (30 μM amylin and 16.2 μM Aβ; 1:1) using amylin-Aβ sandwich ELISAs with the total Aβ antibody as the detection antibody and commercially available amylin antibodies (E-5, Fitzgerald, T4157, Abbexa) or P2 anti-amylin antibody as the capture antibody.

FIGS. 9A-9C. Frequency distributions of amylin, Aβ, and amylin-Aβ hetero-oligomers concentrations in human brain tissues. FIGS. 9A-9C are charts showing frequency distributions as the number of values of Aβ (FIG. 9A) amylin (FIG. 9B), and amylin-Aβ hetero-oligomers (FIG. 9C) concentrations in the brains of APP and PS1 mutation carriers (n=18), sporadic AD (sAD, n=46) and cognitively unaffected (CU; n=13) individuals. Data are means±S.D. Data are presented as histograms in FIGS. 9A-9C.

FIG. 10A is a chart showing residual plot analysis calculated by linear regression for the effect of brain amylin levels on the amylin-Aβ hetero-oligomerization with adjustment for brain Aβ level and a multiplicative (amylin×Aβ) interaction term (see FIG. 5B) in the AD group. FIG. 10B is a chart showing the same as in above for the diabetes group (see FIG. 6B). FIGS. 10C-10D are charts showing pairwise correlation analyses of brain tissue amylin-Aβ versus brain amylin concentrations (FIG. 10C) and brain tissue amylin-Aβ versus Aβ concentrations (FIG. 10D) within the sAD group. Data are presented as correlation analysis, Pearson's correlation.

FIGS. 11A-11F. Characterization of P2 anti-amylin antibody. FIG. 11A is a chart showing P2 amylin antibody titer evaluation by indirect ELISA showing amylin immuno-reactivity signals (normalized to total protein) measured with different dilutions (1:10000 to 1:1000000) of P2 amylin detection antibody. FIG. 11B is a chart showing amylin indirect ELISA showing amylin immuno-reactivity signals (normalized to total protein) measured in different dilutions (200 ng/ml to 3.125 ng/ml) of amylin using the P2 anti-amylin antibody as a detection antibody. FIG. 11C are representative images of immunohistochemistry (IHC) analysis of pancreas sections from HIP (expresses human amylin in pancreas) and AKO (amylin knock out) rats stained with the P2 anti-amylin antibody showing amylin deposition in presumably a pancreatic islet, in a HIP rat. FIG. 11D shows western blot analysis of amylin in pancreatic tissue homogenates from age-matched HIP, wild type (WT), and AKO rats along with synthetic human amylin peptide (70 ng/ml). FIG. 11E shows immunoprecipitation of amylin with P2 anti-amylin antibody and Western blot analysis of amylin with the T4157 anti-amylin antibody in pancreatic tissue homogenates from HIP and AKO rats. FIG. 11F is a chart for amylin indirect ELISA showing amylin immuno-reactivity signal intensities (normalized to total protein) measured in pancreatic tissue homogenates from age matched HIP and AKO rats (n=3 males/group) using P2 and T4157 anti-amylin antibodies. Data are means±S.D. in FIG. 11F; Scale bars 100 μm in FIG. 11C.

### BRIEF DESCRIPTION OF THE SEQUENCE LISTING

SEQ ID NO: 1 is an amino acid sequence for an amylin peptide (Islet Amyloid Polypeptide (IAPP)) which can combine with a beta-amyloid (Aβ) peptide to form an amylin-Aβ aggregate.

SEQ ID NO: 2 is an amino acid sequence for a Aβ peptide which can combine with an amylin peptide to form an amylin-Aβ aggregate.

SEQ ID NO: 3 is an amino acid sequence of an embodiment of an epitope of an amylin-Aβ aggregate to which capture antibody and anti-amylin antibody embodiments disclosed herein may bind.

SEQ ID NO: 4 is an amino acid sequence of an embodiment of an epitope of an amylin-Aβ aggregate to which capture antibody and anti-amylin antibody embodiments disclosed herein may bind.

SEQ ID NO: 5 is an amino acid sequence of an embodiment of an epitope of an amylin-Aβ aggregate to which capture antibody and anti-amylin antibody embodiments disclosed herein may bind.

SEQ ID NO: 6 is an amino acid sequence of an embodiment of an epitope of an amylin-Aβ aggregate to which detection antibody and anti-Aβ antibody embodiments disclosed herein may bind.

SEQ ID NO: 7 is an amino acid sequence which may be present in or have homology to an amino acid sequence of one or more paratopes of capture antibody and anti-amylin antibody embodiments disclosed herein.

SEQ ID NO: 8 is an amino acid sequence which may be present in or have homology to an amino sequence of a paratope of detection antibody and anti-Aβ antibody embodiments disclosed herein.

SEQ ID NO: 9 is an amino acid sequence which may be present in or have homology to an amino sequence of a paratope of detection antibody and anti-Aβ antibody embodiments disclosed herein.

SEQ ID NO: 10 is an amino acid sequence which may be present in or have homology to an amino sequence of a paratope of detection antibody and anti-Aβ antibody embodiments disclosed herein.

### DESCRIPTION OF EXEMPLARY EMBODIMENTS

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

The presently disclosed subject matter is based, in part, on the discovery that certain amino acid residues which are present in amylin-Aβ hetero-oligomers (i.e., amylin-beta amyloid (Aβ) aggregates) and located outside of high affinity binding sites of the amylin and Aβ peptides making up such aggregates provide epitopes which can be targeted in an immunoassay to facilitate the capture, detection, and quantification of amylin-Aβ aggregate within biological samples. The presently disclosed subject matter is further based, in part, on the discovery that a polyclonal anti-amylin antibody and a monoclonal anti-Aβ antibody can be effectively utilized as a capture antibody and a detection antibody, respectively, within a sandwich enzyme-linked immunosorbent assay (ELISA) that can be rapidly scaled to accommodate large sample studies involving amylin-Aβ aggregate detection or quantification. The sandwich ELISA has been found to be effective with respect to the capture, detection, and quantification of amylin-Aβ aggregate present in brain tissue and circulating blood, and to be surprisingly sensitive with respect to distinguishing between amylin-Aβ aggregate and both amylin and Aβ monomers.

Accordingly, in one aspect, the presently disclosed subject matter is directed to methods for detecting or quantifying amylin-Aβ hetero-oligomers (i.e., amylin-Aβ aggregate) which may be present, e.g., in a biological sample of a subject.

In some embodiments, a method for detecting or quantifying amylin-Aβ aggregate involves: contacting a capture antibody that specifically binds to one or more epitopes of amylin-Aβ aggregate with a biological sample in a reaction area (e.g., a well of a microplate); and depositing a detection antibody that specifically binds to an epitope of amylin-Aβ aggregate that is distinct from the one or more epitopes to which the capture antibody specifically binds to within the reaction area. To facilitate the detection and/or quantification of amylin-AB, a detection enzyme may be conjugated (linked) to the detection antibody, where the detection enzyme catalyzes a reaction with a substrate when the detection antibody is bound to amylin-Aβ aggregate. In some embodiments, the method may thus further involve contacting a detection enzyme linked to the detection antibody with a substrate and (i) detecting the presence or absence of amylin-Aβ aggregate within the biological sample based on the presence or absence, respectively, of a signal resulting from a reaction between the substrate and the detection enzyme and/or (ii) quantifying the amount of amylin-Aβ aggregate present within the biological sample based on the intensity of the signal resulting from a reaction between the substrate and detection enzyme.

In some embodiments, a method for detecting or quantifying amylin-Aβ aggregate involves: contacting a capture antibody that specifically binds to one or more epitopes of amylin-Aβ aggregate with a biological sample having amylin-Aβ aggregate present therein to form an initial (or “first”) complex; and contacting a detection antibody with the amylin-Aβ aggregate present in the first complex to form a subsequent (or “second”) complex that facilitates detection and/or quantification of the amylin-Aβ aggregate from the biological sample. The detection antibody specifically binds to an epitope of the amylin-Aβ aggregate from the biological sample that is distinct from the one or more epitopes of the amylin-Aβ aggregate from the biological sample that the capture antibody specifically binds to. To facilitate the detection and/or quantification of the amylin-Aβ aggregate from the biological sample, a detection enzyme may be conjugated (linked) to the detection antibody, where the detection enzyme catalyzes a reaction with a substrate when the detection antibody is bound to the amylin-Aβ aggregate and the detection enzyme is placed in contact with the substrate. Accordingly, in some embodiments, the method further involves contacting the detection enzyme linked to the detection antibody with a substrate and detecting or quantifying the amylin-Aβ aggregate from the biological sample using the signal produced by the reaction between the substrate and the detection enzyme. To immobilize amylin-Aβ aggregate present in the biological sample, the methods disclosed herein may, in some embodiments, further involve coating a surface, such as a solid surface present in a well of a microplate, with the capture antibody. In some embodiments, the capture antibody and the detection antibody of the method may facilitate detection of amylin-Aβ aggregate in concentrations as little as 0.02 ng/mg total protein. In this regard, the amylin-Aβ aggregate from the biological sample can, in some embodiments of the present methods, be characterized as having a limit of detection of 0.02 ng/mg total protein.

In some embodiments, the capture antibody is an anti-amylin antibody that specifically binds to one or more epitopes of amylin peptide present in an amylin-Aβ aggregate. In some embodiments, the anti-amylin antibody specifically binds to one or more epitopes having a sequence selected from KCNTATC (SEQ ID NO: 3), SSN (SEQ ID NO: 4), and STNVGSNTY (SEQ ID NO: 5). In some embodiments, each epitope to which the anti-amylin antibody specifically binds consists of a sequence selected from SEQ ID NO: 3, SEQ ID NO: 4, and SEQ ID NO: 5. In some embodiments, the anti-amylin antibody specifically binds to one or more epitopes within SEQ ID NO: 3, SEQ ID NO: 4, or SEQ ID NO: 5. In some embodiments, the anti-amylin antibody specifically binds to one or more epitopes having homology to SEQ ID NO: 3, SEQ ID NO: 4, or SEQ ID NO: 5.

In some embodiments, the anti-amylin antibody is a polyclonal antibody, which may provide broader recognition and capture of amylin-Aβ aggregate present within the biological sample as compared to, e.g., a monoclonal capture antibody. As the anti-amylin antibody serving as the capture antibody can, in some embodiments, be a polyclonal antibody, it is appreciated that, in such embodiments, the anti-amylin antibody will typically comprise multiple antibodies that include different paratopes that specifically bind to different epitopes of amylin peptide of amylin-Aβ aggregate. Thus, in some embodiments, the anti-amylin antibody may specifically bind to multiple epitopes of an amylin-Aβ aggregate corresponding to amino acid residues of amylin peptide of amylin-Aβ aggregate. In some embodiments, the anti-amylin antibody may include paratopes that comprise or have homology to the amino acid sequence of CKCNTATCATQRLANFLVHSS (SEQ ID NO: 7). In some embodiments, paratopes of the anti-amylin antibody comprise a sequence which is at least 90% homologous to the amino acid sequence of SEQ ID NO: 7. In some embodiments, the anti-amylin antibody specifically binds to a first epitope of an amylin peptide of an amylin-Aβ aggregate comprising SEQ ID NO: 3, a second epitope of the amylin peptide of the amylin-Aβ aggregate comprising SEQ ID NO: 4, and a third epitope of the amylin peptide of the amylin-Aβ aggregate comprising SEQ ID NO: 5. In some embodiments, the anti-amylin antibody specifically binds to a first epitope of an amylin peptide of an amylin-Aβ aggregate consisting of SEQ ID NO: 3, a second epitope of the amylin peptide of the amylin-Aβ aggregate consisting of SEQ ID NO: 4, and a third epitope of the amylin peptide of the amylin-Aβ aggregate consisting of SEQ ID NO: 5. In some embodiments, the anti-amylin antibody specifically binds to a first epitope of an amylin peptide of an amylin-Aβ aggregate having homology to SEQ ID NO: 3, a second epitope of the amylin peptide of the amylin-Aβ aggregate having homology to SEQ ID NO: 4, and a third epitope of the amylin peptide of the amylin-Aβ aggregate having homology to SEQ ID NO: 5.

In some embodiments, the detection antibody is an anti-Aβ antibody that specifically binds to an epitope of amylin-Aβ aggregate that is distinct from the one or more epitopes that the capture antibody specifically binds to. In some embodiments, the anti-Aβ antibody is a monoclonal antibody. In some embodiments, the anti-Aβ antibody is a mid-domain antibody that specifically binds to a mid-region of an Aβ peptide forming part of an amylin-Aβ aggregate. In some embodiments, the anti-Aβ antibody specifically binds to an epitope having a sequence comprising VFFAEDV (SEQ ID NO: 6). In some embodiments, the epitope to which the anti-Aβ antibody specifically binds consists of SEQ ID NO: 6. In some embodiments, the epitope to which the anti-Aβ antibody specifically binds has homology to SEQ ID NO: 6. In some embodiments, the anti-Aβ antibody includes a paratope comprising VFFAE (SEQ ID NO: 8), VGGVVIA (SEQ ID NO: 9), or VGGVV (SEQ ID NO: 10). In some embodiments, the anti-Aβ antibody includes a paratope consisting of SEQ ID NO: 8, SEQ ID NO: 9, or SEQ ID NO: 10. In some embodiments, the anti-Aβ antibody includes a paratope having homology to SEQ ID NO: 8, SEQ ID NO: 9, or SEQ ID NO: 10. In some embodiments, the anti-Aβ antibody is HRP anti-β-amyloid, 17-24 antibody from BioLegend (BioLegend Cat. No. 800720) (total-AB). In some embodiments, the anti-Aβ antibody is HRP anti-β-amyloid, 1-42 antibody from BioLegend (BioLegend Cat. No. 805507) (Aβ₄₂). In some embodiments, the anti-Aβ antibody is HRP anti-β-amyloid, 1-40 antibody from BioLegend (BioLegend Cat. No. 805407) (Aβ₄₀).

In various embodiments, some or all of the epitopes to which the anti-amylin antibody specifically binds is an exposed epitope of amylin peptide of amylin-Aβ aggregate. In some embodiments, the epitope to which the anti-Aβ antibody specifically binds is an exposed epitope of an Aβ peptide of an amylin-Aβ aggregate. Targeting exposed epitopes of the amylin-Aβ increases the sensitivity of the present methods, eliminates background noise, and can facilitate more expeditious optimization of any blocking steps which may be incorporated into the present methods.

In some embodiments, the detection enzyme linked to the detection antibody is horse radish peroxidase (HRP). It is appreciated, however, that other enzymes which can be linked to a capture antibody consistent with those disclosed herein, and which catalyze a reaction with a substrate when the capture antibody is bound to amylin-Aβ aggregate may be utilized in alternative embodiments. For instance, in some embodiments, instead of HRP, alkaline phosphatase (AP) or beta-D-galactosidase may be utilized as the detection enzyme. The substrate utilized in conjunction with the detection enzyme may vary depending on the particular detection enzyme utilized. For instance, in some embodiments the substrate may be selected from tetramethylbenzidine (TMB), o-phenylenediamine dihydrochloride (OPD), 2,2′-azino-bis [3-ethylbenzothiazoline-6-sulfonic acid] (ABTS), para-nitrophenyl phosphate (pNPP), and lactose.

The presence or absence of a signal resulting from the interaction between the detection enzyme and the substrate will ordinarily be indicative of the presence or absence of amylin-Aβ aggregate within the biological sample. As such, detecting amylin-Aβ from the biological sample within the present methods may include inspecting a reaction area (e.g., a well of a microplate) for the presence or absence of a signal indicative of the detection antibody being bound to amylin-Aβ aggregate and the occurrence of a reaction between the detection enzyme and the substrate. The manner in which such inspection is carried out may vary depending on the type of detection enzyme and/or substrate utilized. In this regard, chromogenic (colorimetric), fluorescence, and/or chemiluminescence detection techniques may be employed in various embodiments of the present methods.

The intensity of the signal produced when the substrate is contacted with the detection enzyme will ordinarily be proportional to the amount of the amylin-Aβ aggregate bound by the capture antibody and the detection antibody. Accordingly, in some embodiments, the present methods may involve measuring the signal resulting from the reaction between the detection enzyme and the substrate to quantify amylin-Aβ aggregate present in a biological sample. Measurement of the signal may be facilitated utilizing instruments known and readily available within the art, such as microplate readers, fluorometers, or spectrophotometers. In some embodiments of the present methods, the concentration of amylin-Aβ aggregate in the biological sample may be determined based, at least in part, on the optical density of a sample in which amylin-Aβ aggregate is bound to the capture antibody and the detection antibody.

In some embodiments, the present methods may further include one or more washing and/or incubation steps. For instance, in some embodiments, an incubation step may occur following the coating of the reaction area with the capture antibody, following the deposit of the capture antibody and the biological sample, and/or following the deposit of the detection antibody. In some embodiments, a washing step may occur following deposit of the capture antibody and the biological sample to remove any components unbound to the capture antibody. In some embodiments, the present methods may further involve administering a stop solution to stop the reaction of the detection enzyme and substrate.

The present methods can be used on a wide variety of subjects. Indeed, the term “subject” as used herein is not particularly limited. The term “subject” is inclusive of vertebrates, such as mammals, and the term “subject” can include human and veterinary subjects. Thus, the subject of the herein disclosed methods can be a human, non-human primate, horse, pig, rabbit, dog, sheep, goat, cow, cat, guinea pig, rodent, or the like. The term does not denote a particular age or sex. Thus, adult and newborn subjects, as well as fetuses, whether male or female, are intended to be covered. In some embodiments, the subject from which the biological sample is utilized is a subject suffering from type-2 diabetes. In some embodiments, the subject from which the biological sample was obtained may be diagnosed with type-2 diabetes. In some embodiments, the subject from which the biological sample is utilized is a subject suffering from Alzheimer's disease (AD). In some embodiments, the subject from which the biological sample is obtained may be diagnosed with AD.

With regard to the step of providing or utilizing a biological sample from a subject, the term “biological sample” as used herein refers to any body fluid or tissue which may contain amylin-Aβ aggregate. In some embodiments, for example, the biological sample can be a blood sample, a serum sample, a plasma sample, or sub-fractions thereof. In some embodiments, the biological sample comprises a brain tissue sample.

In some embodiments of the present methods, the amylin-Aβ aggregate within the biological sample is prefibrillar amylin-Aβ aggregate. Accordingly, in some embodiments, the present methods include detecting and/or quantifying prefibrillar amylin-Aβ aggregate from the biological sample.

Reagents utilized in the methods disclosed above can be provided in commercial kits that facilitate amylin-Aβ aggregate detection and/or quantification. Accordingly, in another aspect, the presently disclosed subject matter includes kits useful in amylin-Aβ aggregate detection and/or quantification.

A kit in accordance with the present disclosure can include: an anti-amylin antibody that specifically binds to one or more epitopes of amylin-Aβ aggregate; and an anti-Aβ antibody that specifically binds to an epitope of the amylin-Aβ aggregate that is distinct from the one or more epitopes of the amylin-Aβ aggregate that the anti-amylin antibody specifically binds to. The same antibodies as described above for the capture antibody and anti-amylin antibody in the various embodiments of the methods for detecting or quantifying amylin-Aβ aggregate may be utilized as the anti-amylin antibody in various embodiments of the kit. The same antibodies as described above for the detection antibody and anti-Aβ antibody in the various embodiments of the methods for detecting or quantifying amylin-Aβ aggregate may be utilized as the anti-Aβ antibody in various embodiments of the kit. All combinations of the various anti-amylin antibody embodiments and various anti-Aβ antibody embodiments described above for the methods for detecting or quantifying amylin-Aβ aggregate are contemplated.

In some embodiments, the kit further includes at least one of: a detection enzyme linked to the anti-Aβ antibody; a substrate; a microplate including a plurality of wells; washing buffer; and stop solution that terminates the reaction between the substrate and the detection enzyme. Detection enzymes and substrates which may be used include those described above in the various embodiments of the methods for detecting or quantifying amylin-Aβ aggregate.

While the terms used herein are believed to be well understood by those of ordinary skill in the art, certain definitions are set forth to facilitate explanation of the presently disclosed subject matter.

Unless defined otherwise, all technical and scientific terms used herein have the same meaning as is commonly understood by one of skill in the art to which the invention(s) belong.

All patents, patent applications, published applications and publications, GenBank sequences, databases, websites and other published materials referred to throughout the entire disclosure herein, unless noted otherwise, are incorporated by reference in their entirety.

Where reference is made to a URL or other such identifier or address, it is understood that such identifiers can change and particular information on the internet can come and go, but equivalent information can be found by searching the internet. Reference thereto evidences the availability and public dissemination of such information.

As used herein, the abbreviations for any protective groups, amino acids and other compounds, are, unless indicated otherwise, in accordance with their common usage, recognized abbreviations, or the IUPAC-IUB Commission on Biochemical Nomenclature (see, Biochem. (1972) 11 (9): 1726-1732).

Although any methods, devices, and materials similar or equivalent to those described herein can be used in the practice or testing of the presently disclosed subject matter, representative methods, devices, and materials are described herein.

In certain instances, nucleotides and polypeptides disclosed herein may be included in publicly-available databases, such as GENBANK and SWISSPROT. Information including sequences and other information related to such nucleotides and polypeptides included in such publicly-available databases are expressly incorporated by reference. Unless otherwise indicated or apparent, the references to such publicly-available databases are references to the most recent version of the database as of the filing date of this Application.

The present application can “comprise” (open ended) or “consist essentially of” the components of the present invention as well as other ingredients or elements described herein. As used herein, “comprising” is open ended and means the elements recited, or their equivalent in structure or function, plus any other element or elements which are not recited. The terms “having” and “including” are also to be construed as open ended unless the context suggests otherwise.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently disclosed subject matter.

As used herein, the term “about,” when referring to a value or to an amount of mass, weight, time, volume, concentration or percentage is meant to encompass variations of in some embodiments±20%, in some embodiments±10%, in some embodiments±5%, in some embodiments±1%, in some embodiments±0.5%, in some embodiments±0.1%, in some embodiments±0.01%, and in some embodiments±0.001% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, “optional” or “optionally” means that the subsequently described event or circumstance does or does not occur and that the description includes instances where said event or circumstance occurs and instances where it does not. For example, an optionally variant portion means that the portion is variant or non-variant.

As used herein, “amylin-beta amyloid (Aβ) hetero-oligomer”, “amylin-AB”, and “amylin-beta amyloid (Aβ) aggregate” are used interchangeably and refer to an aggregate that comprises an amylin peptide and a Aβ peptide bound together at one or more binding sites along the amylin peptide and the Aβ peptide.

As used herein, an “exposed epitope” in the context of an amylin peptide of an amylin-Aβ aggregate refers to a region of the amylin peptide that is not bound to a Aβ peptide of the amylin-Aβ aggregate. Similarly, an “exposed epitope” in the context of a Aβ peptide of an amylin-Aβ aggregate refers to a region of the Aβ peptide that is not bound to an amylin peptide of the amylin-Aβ aggregate.

An “antibody” is an immunoglobulin molecule capable of specific binding to a target, such as a carbohydrate, polynucleotide, lipid, polypeptide, etc., through at least one antigen recognition site, located in the variable domain of the immunoglobulin molecule. As used herein, the term encompasses not only intact polyclonal or monoclonal antibodies, but also, unless otherwise specified, any antigen-binding portion thereof that competes with the intact antibody for specific binding, fusion proteins comprising an antigen-binding portion, and any other modified configuration of the immunoglobulin molecule that comprises an antigen recognition site. Antigen-binding portions include, for example, Fab, Fab′, F(ab′) 2, Fd, Fv, domain antibodies (dAbs, e.g., shark and camelid antibodies), portions including complementarity determining regions (CDRs), single chain variable fragment antibodies (scFv), maxibodies, minibodies, intrabodies, diabodies, triabodies, tetrabodies, v-NAR and bis-scFv, and polypeptides that contain at least a portion of an immunoglobulin that is sufficient to confer specific antigen binding to the polypeptide. Depending on the antibody amino acid sequence of the constant region of its heavy chains, immunoglobulins can be assigned to different classes. There are five major classes (i.e., isotypes) of immunoglobulins: IgA, IgD, IgE, IgG, and IgM, and several of these may be further divided into subclasses (subtypes), e.g., IgG1, IgG2, IgG3, IgG4, IgA1 and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

As used herein, an “isolated” or “purified” polypeptide or protein (e.g. an isolated antibody or antigen-binding fragment thereof) or biologically-active portion thereof (e.g. an isolated antigen-binding fragment) is substantially free of cellular material or other contaminating proteins from the cell or tissue from which the protein is derived, or substantially free from chemical precursors or other chemicals when chemically synthesized. Preparations can be determined to be substantially free if they appear free of readily detectable impurities as determined by standard methods of analysis, such as thin layer chromatography (TLC), gel electrophoresis and high performance liquid chromatography (HPLC), used by those of skill in the art to assess such purity, or sufficiently pure such that further purification does not detectably alter the physical and chemical properties, such as enzymatic and biological activities, of the substance. The antibodies utilized in the methods and kits of the present disclosure can be isolated antibodies.

The terms “antigen-binding portion” or “antigen-binding fragment” of an antibody (or simply “antibody portion”), as used interchangeably herein, refers to one or more portions of an antibody that retain the ability to specifically bind to an antigen. It has been shown that the antigen-binding function of an antibody can be performed by portions of a full-length antibody.

A “variable domain” of an antibody refers to the variable domain of the antibody light chain (V_{L}) or the variable domain of the antibody heavy chain (V_{H}), either alone or in combination. As known in the art, the variable domains of the heavy and light chains each consist of four framework regions (FRs) connected by three complementarity determining regions (CDRs) also known as hypervariable regions, and contribute to the formation of the antigen-binding site of antibodies. A “paratope” is a site on an antibody within the variable region that recognizes and binds to a specific antigen, and includes contributions from the V_{H} and V_{L} CDRs.

As used herein, “monoclonal antibody” refers to an antibody obtained from a population of substantially homogeneous antibodies, i.e., the individual antibodies comprising the population are identical except for possible naturally-occurring mutations that may be present in minor amounts. Monoclonal antibodies are highly specific, being directed against a single antigenic site. Furthermore, in contrast to polyclonal antibody preparations, which typically include different antibodies directed against different determinants (epitopes), each monoclonal antibody is directed against a single determinant on the antigen. The modifier “monoclonal” indicates the character of the antibody as being obtained from a substantially homogeneous population of antibodies, and is not to be construed as requiring production of the antibody by any particular method.

The term “antigen (Ag)” refers to the molecular entity used for immunization of an immunocompetent vertebrate to produce the antibody (Ab) that recognizes the Ag or to screen an expression library (e.g., phage, yeast or ribosome display library, among others). Herein, Ag is termed more broadly and is generally intended to include target molecules that are specifically recognized by the Ab, thus including portions or mimics of the molecule used in an immunization process for raising the Ab or in library screening for selecting the Ab.

Generally, the term “epitope” refers to the area or region of an antigen to which an antibody specifically binds, i.e., an area or region in physical contact with the antibody. Thus, the term “epitope” refers to that portion of a molecule capable of being recognized by and bound by an antibody at one or more of the antibody's antigen-binding regions.

As used herein, when a polypeptide molecule or region thereof contains or has “homology” to another polypeptide molecule or region, the two molecules and/or regions share greater than or equal to at or about 40% sequence identity, and typically greater than or equal to at or about 50% sequence identity, such as at least or about 60%, 65%, 70%, 75%, 80%, 85%, 90%, 95%, 96%, 97%, 98%, 99% or 100% sequence identity; the precise percentage of identity can be specified if necessary. “Percent homology” when used herein to describe to an amino acid sequence or a nucleic acid sequence, relative to a reference sequence, can be determined using the formula described by Karlin and Altschul (Proc. Natl. Acad. Sci. USA 87:2264-2268, 1990, modified as in Proc. Natl. Acad. Sci. USA 90:5873-5877, 1993).

The term ““homology” when associated with a particular number, represents a comparison between the sequences of a first and a second polypeptide or polynucleotide or regions thereof and/or between theoretical nucleotide or amino acid sequences. For instance, the term “at least 90% homologous to” refers to percent identities from 90 to 99.99 relative to the first nucleic acid molecule or amino acid sequence of the polypeptide. Identity at a level of 90% or more is indicative of the fact that, assuming for exemplification purposes, a first and second polypeptide length of 100 amino acids are compared, no more than 10% (i.e., 10 out of 100) of the amino acids in the first polypeptide differs from that of the second polypeptide. Similar comparisons can be made between first and second polynucleotides. Such differences among the first and second sequences can be represented as point mutations randomly distributed over the entire length of a polypeptide or they can be clustered in one or more locations of varying length up to the maximum allowable, e.g. 10/100 amino acid difference (approximately 90% identity). Differences are defined as nucleotide or amino acid residue substitutions, insertions, additions or deletions.

An antibody that “specifically binds” to an epitope is a term well understood in the art, and methods to determine such specific or preferential binding are also well known in the art. A molecule is said to exhibit “specific binding” if it reacts or associates more frequently, more rapidly, with greater duration and/or with greater affinity with a particular cell or substance than it does with alternative cells or substances. An antibody “specifically binds” to a target if it binds with greater affinity, avidity, more readily, and/or with greater duration than it binds to other substances. Also, an antibody “specifically binds” to a target if it binds with greater affinity, avidity, more readily, and/or with greater duration to that target in a sample than it binds to other substances present in the sample. As such, “specific binding” does not necessarily require (although it can include) exclusive binding.

The presently disclosed subject matter is further illustrated by the following specific but non-limiting examples. The following examples may include compilations of data that are representative of data gathered at various times during the course of development and experimentation related to the present disclosure.

### EXAMPLE

Alzheimer's disease (AD) is associated with dysregulated pancreatic hormones such as insulin leading to brain insulin resistance that can exacerbate AD pathology. Amylin, a pancreatic B-cell hormone co-synthesized and co-secreted with insulin, crosses from the blood to the brain parenchyma, and is involved in the central regulation of satiation. In persons with type-2 diabetes mellitus, amylin forms pancreatic amyloid (>95% prevalence at autopsy) which contributes to type-2 diabetes pathogenesis by inducing β-cell apoptosis and β-cell mass depletion. Amylin also synergistically co-aggregates with vascular and brain parenchymal Aβ in the brain in both sporadic and early-onset familial AD. Using APPswe/PS1dE9 (APP/PS1) rats expressing human amylin specifically in the pancreas (murine amylin is non-amyloidogenic), chronic exposure to circulating amyloid-forming amylin was found to promote cerebrovascular and parenchymal amylin-Aβ deposition, consistent with findings in human AD brains. These results suggest an association between pancreatic amyloid-forming amylin and Aβ pathology. Whether co-aggregation of amylin and Aβ is the mechanism underlying this association, however, remains unknown.

Although the need to quantify molecular amylin-Aβ interaction in human AD has been recognized, the complexity and lack of scalability of traditional methods (such as immunoprecipitation, Western blot, circular dichroism, and electron microscopy) have hampered studies requiring larger sample sizes. For example, studies of amylin-Aβ hetero-oligomers in human brain homogenates by co-immunoprecipitation and Western blot were conducted in only a few samples. Furthermore, denaturing conditions present in SDS gels may alter the structural stability and size of the amylin-Aβ hetero-oligomer, unlike other methods such as ELISA in which proteins remain in their native state. The levels of amylin-Aβ hetero-oligomers in the brains of humans with AD compared to those in the brains of unaffected persons remain unknown.

The studies underlying this example were carried out to develop an ELISA to detect amylin-Aβ hetero-oligomers in brain tissue and blood. Using the developed assay, human temporal cortex homogenates for amylin-Aβ hetero-oligomers were screened and transgenic AD-model rats were used to detect circulating amylin-Aβ hetero-oligomers in the blood. Direct assessment of co-aggregated amylin-Aβ in brain tissues and blood may help to better delineate the mechanisms underlying the association between pancreatic amyloid-forming amylin and cerebral Aβ pathology. This is important because therapeutic strategies to block amylin-Aβ co-aggregation could reduce or delay the development and progression of AD.

### Methods

### Amylin-Aβ Co-Aggregatin

The amino acid sequences of amylin and Aβ peptides promoting amylin-Aβ co-aggregation (i.e., formation of amylin-Aβ hetero-oligomers) were assessed by using synthetic amylin and Aβ peptides. Heterologous seeding between amylin and various Aβ fragments (i.e., Aβ₄₂ and Aβ₄₀) propagates amyloid formation comparable to that of homogenous amylin amyloid. Co-expression of the two peptides in cells promotes in vivo amylin-Aβ hetero-amyloid formation and cytotoxicity. Immunoprecipitated amylin from human AD brain tissue analyzed by Western blot using an anti-Aβ antibody detected Aβ immunoreactivity indicates that the amylin and Aβ peptides aggregate in the human AD brain forming amylin-Aβ hetero-oligomers that are sodium dodecyl sulfate (SDS)-soluble. These data collectively confirm that amylin-Aβ hetero-oligomers can be detected and quantified, and may serve as a marker of amylin-Aβ established interaction and its correlation with AD pathology.

### Anti-Amylin Antibody Generation

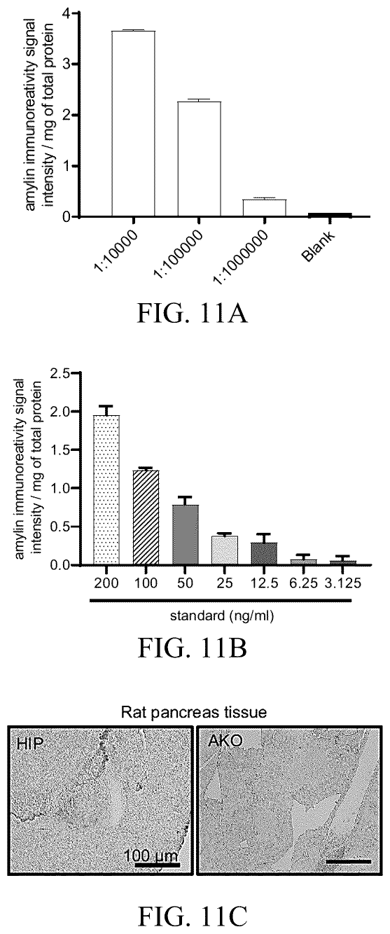

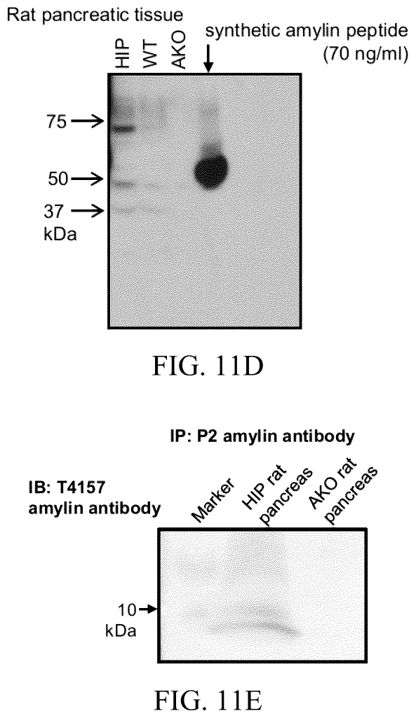

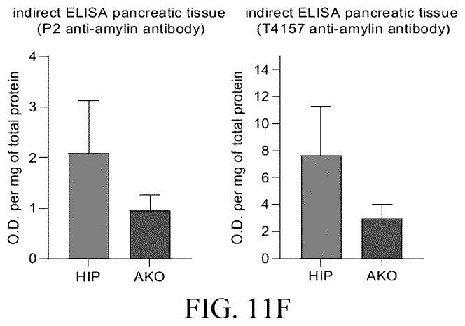

Immunization of New Zealand white rabbit was performed with purified N-terminal synthetic amylin peptide (5′-CKCNTATCATQRLANFLVHSS-3′) conjugated with Keyhole limpet hemocyanin (KLH) antigen as described by Saradhi et al. with some modifications. Briefly, the rabbit was immunized subcutaneously at four different sites with 1 mg of immunogen after suspending in equal volume of complete Freund's adjuvant (0.5 ml). Every three weeks after initial immunization, the rabbit was injected with 1 mg of amylin with Alum adjuvant as booster injections for a total of three booster doses administered. Pre-immunization blood was collected before the first dose of injection and before every booster dose from the rabbit's central artery of ear under sedation. Three weeks after the final booster dose, blood was collected by cardiac puncture under full anesthesia. The blood serum was isolated and further purified by Protein A Plus Spin Columns (NAb™, 89956, Thermo Scientific) followed by characterization of P2 amylin antibody with immunoprecipitation (IP), Western blot, ELISA, and immunohistochemistry. The characterization of the anti-amylin P2 antibody is detailed in FIG. 11.

### Amylin-Aβ Sandwich ELISA

Amylin-Aβ sandwich ELISA was used to measure the concentrations of amylin-Aβ in human/rat brain homogenates. Briefly, a 96 well plate was coated with 100 μL of mouse anti-human-Aβ (1:400; clone 6E10, 803002, Biolegend) antibody for standard and with rabbit anti-amylin P2 antibody (1:400; 2 mg/ml stock) for samples in bicarbonate buffer (0.028 M Na₂CO₃, 0.071 M NaHCO₃, pH 9.6) as coating antibodies overnight at 4C temperature. The next day, plate was washed 1-2 times with 300 μL of washing buffer [Phosphate Buffer Saline with 0.05% of Tween-20 (0.05% PBST)] and blocked by 300 μL of assay diluent (421203, Biolegend) for 1 hour at room temperature. Again, the plate was washed with 0.05% PBST washing buffer and incubated with 100 μL of Aβ₄₀/Aβ₄₂ standards and brain homogenate samples overnight at 4C. Assay diluent/PBS was used as a blank for standard and sample wells. The next day after three washing cycles, the plate was incubated with 100 μL of either mouse anti-human-total-Aβ (1:400, clone-4G8, 800720-Biolegend), mouse anti-human-Aβ₄₂ (1:400, 805507, Biolegend; specific for the isoform ending at the 42^{nd} amino acid) or mouse anti-human-Aβ₄₀ (1:400, 805407, Biolegend; specific for the isoform ending at the 40th amino acid) conjugated-HRP detection antibody for 1 hour at room temperature. After completing the incubation, the plate was washed 3-4 times followed by incubation with 100 μL of 3,3′,5,5′ tetramethylbenzidine (TMB) substrate (34028, Thermo Scientific). The reaction was stopped by adding 50 μL of stop solution (N600, Thermo Scientific) after developing the signals, the plate was read at 450 nm in a spectrophotometer. Amylin-Aβ concentrations were calculated by generating a graph between relative optical density (O.D.) 450 of standards vs standard concentrations, after subtracting the optical density of standard blank and sample blank from the optical density of standards and samples respectively.

The limit of detection (LOD) of the amylin-Aβ sandwich ELISA (0.02 ng/mg of total protein) is calculated as reported by Armbruster et al. (41). LOD is the lowest analyte concentration likely to be reliably distinguished from the limit of blank (LOB) at which detection is feasible. LOD is determined by utilizing both the measured LOB and test replicates of a sample known to contain a low concentration of analyte: LOD=LOB+1.645*(SD low concentration sample). LOB is the highest apparent analyte concentration expected to be found when replicates of a blank sample containing no analyte are tested: LOB=mean blank+1.645*(SD blank), where SD is the standard deviation.

In a separate experiment commercially available amylin antibodies (1:400, T-4157, Bachem-Peninsula Laboratories, CA; 1:400, SC-377530, E5 Santa Cruze; 1:400, abx-324813, Abbexa; 1:400, 70R-15263, Fitzgerald) were used as coating antibody to compare with the rabbit anti-amylin P2 antibody in amylin-Aβ sandwich ELISA.

### Conventional Amylin and Aβ ELISAS

Indirect ELISA was used to measure the concentrations of amylin in human brain, rat brain, and pancreas homogenates as described by Kohl et al. with some modifications. Briefly, a 96-well plate was coated with 50 μL of bicarbonate buffer with 50 μL of amylin standard and brain homogenate samples overnight at 4° C. temperature. The next day, the plate was washed with 300 μL of washing buffer [Tris Buffer Saline with 0.05% of Tween-20 (0.05% TBST)] and blocked by 300 μL of assay diluent for 1 hr. at room temperature. Again, the plate was washed with 0.05% TBST washing buffer and incubated with 100 μL volume of rabbit anti-amylin P2 detection antibody (1:400) overnight at 4° C. temperature. The next day, the plate was washed three times with washing buffer and incubated with anti-rabbit IgG HRP-conjugated (1:400; NA934VS; GE Healthcare) antibody for 1 hr. at room temperature. The plate was washed 3-4 times followed by incubation with 100 μL of TMB substrate. The reaction was stopped by adding 50 μL of stop solution after getting the signals and the plate was read at 450 nm in a spectrophotometer. For comparison, a commercially-available rabbit anti-amylin antibody (1:400, T-4157, Bachem-Peninsula Laboratories, CA) was also used as a detection antibody in rat pancreas homogenate.

To measure the titer of rabbit anti-amylin P2 antibody, an indirect ELISA was used. The end-point dilution method was used to determine the titer of amylin P2 antibody. Antibody titers were defined as the reciprocal of the highest amylin P2 antibody dilution that produced an optical density value above the blank.

Amylin ELISA (EZHA-52K, Millipore Sigma) was used to measure the amylin concentrations in brain homogenates. Aβ₄₂ concentrations in brain homogenates were measured using the sandwich ELISA Kit for Aβ₄₂ (Thermo Fisher Scientific, cat #KHB3441). Concentrations of target proteins were normalized to the amount of total protein input assessed using the BCA method in all experiments.

### Human Samples

This research employed de-identified frozen brain tissue from the biobank of the Alzheimer's Disease Research Center at the University of Kentucky (UK-ADRC) under a protocol approved by the University of Kentucky Institutional Review Board (IRB). Informed consent was obtained prospectively. Frozen temporal cortex tissue samples were obtained from 45 persons with sporadic (SAD)-type dementia documented by Aβ positivity and 13 cognitively unaffected individuals. Patient characteristics including cognitive status, sex, diabetes status, and age, along with clinical and neuropathological information are described in Table 1.

TABLE 1
Summary statistics for individuals with sAD

| | AD | Non-AD |
|---|---|---|
| Brain samples | n = 45 | n = 13 |
| (ELISA) | | |
| Gender, female/male | 23/22 | 10/3 |
| (% female) | (51.1) | (76.92) |
| Age at collection in | 85.65 ± 7.04 | 87.22 ± 7.30 |
| years (avg ± SEM) | | |
| Type-2 diabetes (%) | 51.06 | 38.46 |

The absence/presence of diabetes was determined during life (at longitudinal clinical visits) by patient or caregiver self-report and the use of diabetic medications. The assessment of clinical dementia and the neuropathologic features-neuritic amyloid plaques (Consortium to Establish a Registry for Alzheimer's Disease; CERAD) and Braak NFT stage—were scored as previously described. Amylin-Aβ concentrations were measured in familial AD (fAD) brains (n=18) with previously documented amylin accumulation through IHC and ELISA. Frozen temporal cortex tissues from fAD mutation carriers were provided by the Queen Square Brain Bank for Neurological Disorders at UCL Queen Square Institute of Neurology (United Kingdom) and King's College London (United Kingdom). Patient characteristics were previously described.

### Experimental Animals

This investigation conforms to the Guide for the Care and Use of Laboratory Animals published by the US National Academies Press (8th edition, 2011) and was approved by the Institutional Animal Care and Use Committee at the University of Kentucky. Transgenic rats (n=26) and rabbits (all males) were used. New Zealand white rabbits aged 2-3 months (Charles River Labs; n=2) were used for immunization to generate rabbit anti-amylin P2 polyclonal antibody. Rats were housed in individually ventilated cages, on a 12-hour light cycle and received a standard pelleted diet and water ad libitum. APP/PS1/HIP rats (n=10 males; age 16 months) with APP/PS1 rats (n=10 males; age 16 months) were used in measurements of amylin-Aβ concentrations in brain tissues and blood by using the amylin-Aβ sandwich ELISA. APP/PS1/HIP rats were generated as previously described. Briefly, TgF344-19 rats from the Rat Resource and Research Center, Univ. of Missouri, Columbia, MO (APP/PS1 rats) are Fischer rats that express human Aβ (A4) precursor protein (hAPP) gene with the Swedish mutation (K595N/M596L), and presenilin 1 (PSEN1) gene with a deletion of exon 9, driven by mouse prion promoter (Prp). The APP/PS1 rats were crossbred with HIP rats to generate rats that are triple transgenic for human amylin, APP, and PSEN1 (APP/PS1/HIP rats). APP/PS1 rats that express non-amyloidogenic rat amylin served as controls for the amyloidogenic human amylin effects. Amylin knockout (AKO) rats that were characterized previously were used as negative controls for amylin in biochemical studies.

### Tissue Homogenization

Frozen human brain, rat brain, and pancreas tissues were homogenized in homogenate buffer (150 mM NaCl, 50 mM Tris-HCl, 50 mM NaF, 2% Triton X-100, 0.1% SDS, 1% (v/v) protease and phosphatase inhibitors, pH 7.5). The homogenates were left on ice for 15 minutes. Homogenates were centrifuged at 12,000×g for 20 minutes at 4° C. The supernatant was separated from pellet after centrifugation and was then used for all experiments. For monomeric amylin, 100 μL of rat brain homogenates supernatants were treated with 20-30 μL of 1M Hydrochloric (HCl) acid for 30 min at room temperature and then incubated on ice for 15 min. By adding 5-10 μL 1M Tris pH was neutralized and used in amylin-Aβ sandwich ELISA.

### Amylin-Aβ Aggregate Formation and the Thioflavin T Assay for Amyloid

Amylin-Aβ aggregates were prepared using the procedure described by O'Nuallain et al. Briefly, 30 μM human amylin synthetic peptide (AS-60254-1, Anaspec) was incubated in PBSA (PBS+0.05% sodium azide) in a water bath at 37° C. for 5 hrs. Aβ₄₀ synthetic peptide (16.2 μM) (AS-24235, Anaspec) was preincubated at 37° C. for 2 hrs. The two solutions (1:1 volume) were then incubated in a water bath at 37° C. for 24 hrs. Aggregated amylin-Aβ was used in amylin-Aβ sandwich ELISA.

For Thioflavin T assay, 10 μL of solutions of prepared peptide aggregates were added in 96 well plate with 100 μL of 0.08 mg/mL Thioflavin T (Sigma). The plate was incubated at 37° C. for one min and fluorescence at 440 nm excitation and 482 nm emission was measured.

### Immunohistochemistry and Immunofluorescence

For immunohistochemistry, formalin-fixed, paraffin-embedded brain tissues from HIP and AKO rats were processed as described before (12, 43). Rabbit anti-amylin P2 antibody was the primary antibody (dilution 1:100). Biotinylated anti-rabbit IgG (1:300, BA-1100, Vector) was the secondary antibody. Pancreas tissues from AKO rats were the negative control for amylin.

In immunofluorescence experiments, formalin-fixed, paraffin-embedded human brain tissue processed as previously described (12, 36, 43, 44) was used. Anti-amylin (1:200; clone E5; SC-377530; Santa Cruz, and 1:200; T-4157, Bachem-Peninsula Laboratories), anti-Aβ (1:400; clone 6E10, 803002, Biolegend), were the primary antibodies. Secondary antibodies were: Alexa Fluor 488 conjugated anti-mouse IgG (1:300; A11029; Invitrogen), Alexa Fluor 568 conjugated anti-rabbit IgG (1:200; A11036; Invitrogen), Alexa Fluor 647 conjugated anti-mouse (#A21236, Invitrogen) and Alexa Fluor 568 conjugated anti-mouse IgG (1:300; A11004; Invitrogen). For Thioflavin S staining, after secondary antibody incubation, brain slides were incubated in 0.5% Thioflavin S for 30 minutes at room temperature. Slides were then incubated for 3 minutes in 70% ethanol, 5 minutes in 0.2% Sudan black before washing and mounting. Immunofluorescence was performed as described previously.

### Proximity Ligation Assay

Formalin-fixed and paraffin-embedded sections (10 μm) from brain were rehydrated and pretreated with 95% formic acid for 3 minutes at ambient temperature to expose antigens. After rinsing in 50 mmol/L Tris-HCl buffer with 150 mmol/L NaCl (pH 7.4), brain sections were incubated with primary antibodies rabbit anti-human-amylin (1:200, anti-human-amylin; T-4157; Peninsula laboratories), and mouse anti-Aβ antibody 6E10 (1:200, 803002, Biolegend) overnight at 4° C. Duolink in situ PLA (Duolink in situ PLA, DUO92004, Sigma, USA) was performed according to the manufacturer's protocol and previously described in detail (10, 44). Briefly, for detection of primary antibody pairs, sections were incubated for 90 minutes with oligonucleotide-conjugated anti-mouse IgG MINUS and anti-rabbit IgG PLUS (PLA probes) diluted 1:6 in Tris-buffered saline at 37° C. Amplified DNA strands were detected with oligonucleotides conjugated to a fluorophore, and nuclei stained with Hoechst dye.

### Immunoprecipitation

A previously published protocol was used. Briefly, rat pancreas homogenates (1000 μg of total protein) and amylin-Aβ (30 μM amylin and 16.2 μM Aβ; 1:1) aggregates were incubated with rabbit anti-amylin P2 antibody (1:25, 2 mg/ml stock) and normal rabbit Ig G (#2729; Cell Signaling Technology) as a control, overnight with end-over-end rotation, at 4° C. Antigen-antibody complex was added to Immobilized Protein A/G resin slurry (20422, Thermo Scientific) for 2 hours at ambient temperature, washed with wash buffer (5 mM of EGTA, 50 mM of Tris, 1% v/v of Triton X-100, pH 7.5+1% v/v protease inhibitor, 1% v/v phosphatase inhibitor) and samples eluted with elution buffer (1.5% w/v of Glycine, 8% v/v of 1 N HCl, pH 2-3) from the resins using elution buffer. The eluate was used for Western blot analysis.

### Western Blot

Western blot analysis was performed on amylin-Aβ IP eluates, rat pancreas IP eluates, and pancreas tissue homogenate from rats. Tissues were processed as described previously. Briefly, rat brain/pancreas tissue homogenates were prepared in homogenate buffer. The homogenates were left on ice for 15 minutes, and centrifuged at 12,000×g for 20-minutes. The supernatant was separated from the pellet after centrifugation and then used for Western blotting. Total protein levels were estimated using a BCA kit (23225, ThermoFisher). Rabbit anti-amylin P2 (1:400), Rabbit anti-amylin polyclonal (1:2,000; T-4157, Bachem-Peninsula Laboratories, CA), mouse anti human-Aβ (1:400; clone 6E10, 803002, Biolegend) antibody were primary antibodies. Rat brain homogenates or immunoprecipitated rat amylin from pancreas homogenates or amylin-Aβ aggregates IP eluates (50 μg of protein from tissue homogenate or immunoprecipitated amylin elution) were loaded on 8% SDS-PAGE gels. Amylin was resolved in SDS-PAGE. anti-rabbit IgG HRP conjugated (1:30,000; NA934VS; GE Healthcare), and anti-mouse IgG HRP-conjugated (1:20,000; NXA931; GE Healthcare) were secondary antibodies.

### Statistical Analysis

The number of samples or animals in each analysis, the statistical analysis performed, and P values are reported in figures and figure legends. D'Agostino-Pearson and Kolmogorov-Smirnov test was used to test the normality distribution of continuous variables. Parametric comparisons of continuous variables with normal distributions were performed using two-tailed unpaired t-test. Welch's correction was used with t-test to account for unequal variance from unequal sample sizes, if necessary. Parametric comparisons of three groups or more group means were performed using one-way or two-way ANOVA with the Bonferroni post-test. Relationships between two continuous variables were analyzed by correlation analysis. Data are presented as mean±S.D. or as box and whisker plots. Difference between groups was considered significant when P. 0.05. Part of analyses were performed using GraphPad Prism 8.1 software.

### Regression Models

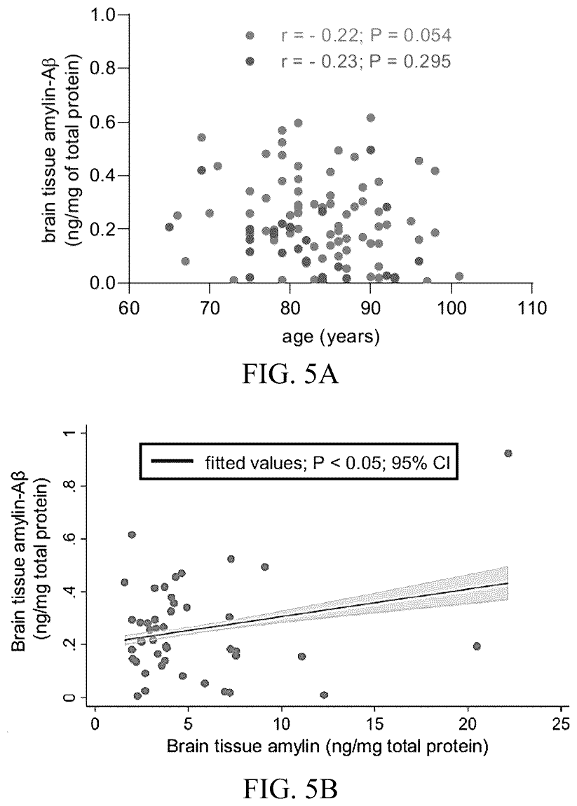

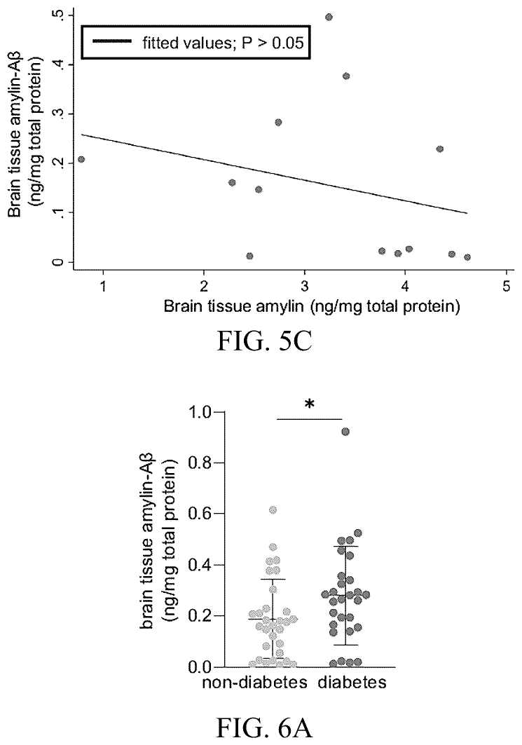

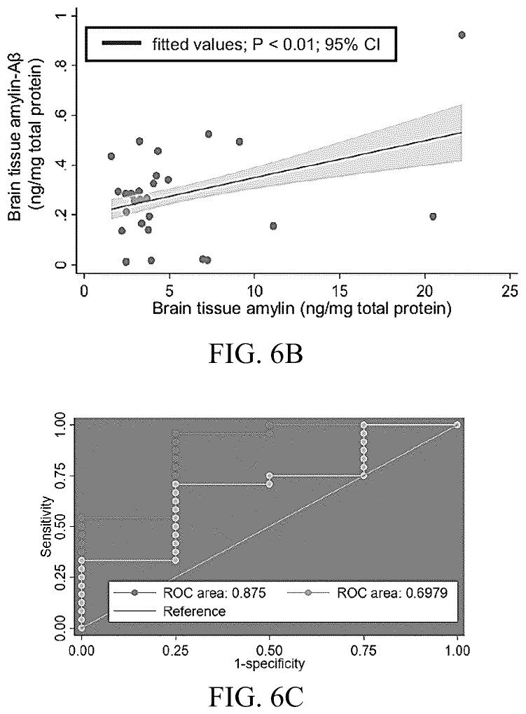

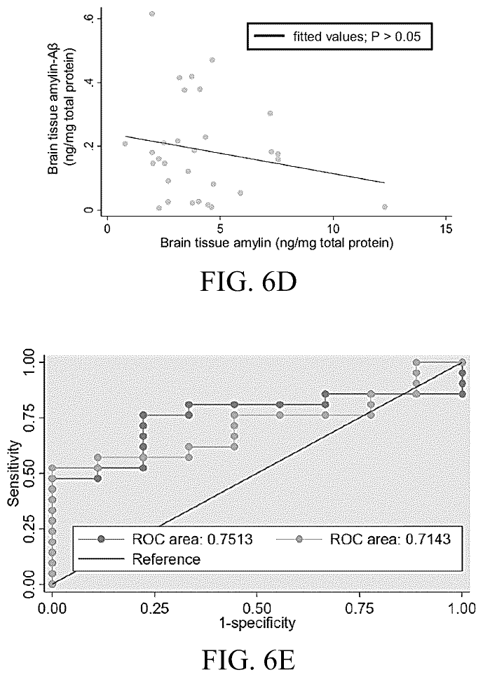

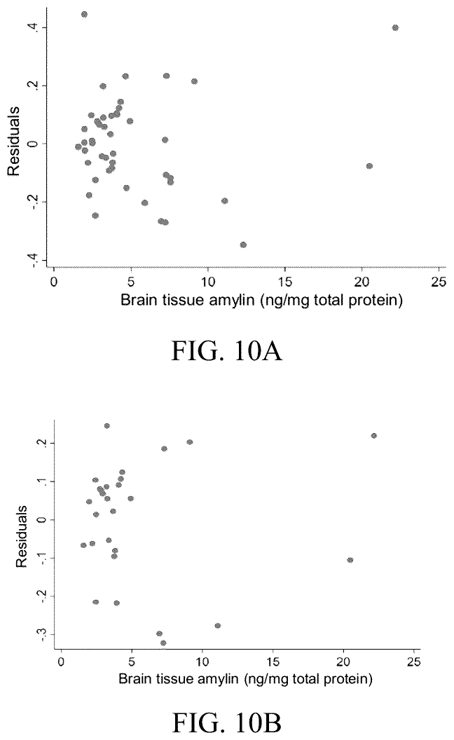

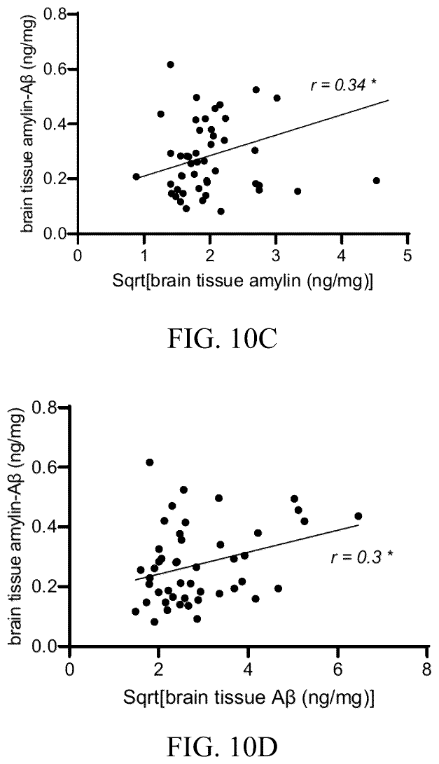

STATA BE-17 was used for conducting multiple linear and logistic regression models. We employed regression analyses of the relationships between amylin-Aβ hetero-oligomerization and brain amylin and Aβ levels (predictor variables) in persons stratified on diagnosis of AD versus non-AD and diabetes versus non-diabetes. Age and sex were included as covariates in all analyses. To assess possible multicollinearity, the variance Inflation Factor (VIF) post estimation test was conducted for each multiple linear regression model. Models with VIF>10 were discarded. In our multiple linear regression model (FIG. 6B), the mean VIF is 4.19 indicating moderate correlation between the amylin and Aβ predictor variables. Residual plots were calculated for all models to detect outliers, assess homoscedasticity and need for transformations. In the linear regression analyses, including age as a covariate leads to no significant change of the regression coefficients (P>0.05); same for the sex covariate (P>0.05). Residual plots did not detect significant outliers (FIGS. 10A-10B); however, transformations of amylin and Aβ covariates (FIGS. 10C-10D) may improve the two linear regression models shown in FIG. 5B and FIG. 6B.

The logistic regression models included age and brain amylin, Aβ and amylin-Aβ levels as covariates and were used for generating Receiver Operating Characteristic (ROC) curves and calculating the areas under the curves. The Hosmer-Lemeshow goodness of fit was used to test the fit in each model. Akaike's information criterion and Bayesian information criterion were used to compare the models. For example, in FIG. 6C, STATA calculated AIC=23.18 and BIC=28.50 for the logistic regression model corresponding to amylin-Aβ-based model and AIC=26.75 and BIC=30.75 for the logistic regression model corresponding Aβ-based model, indicating the superiority of the former.

### Results

### A Specific ELISA for Measuring Amylin-Aβ Hetero-Oligomers in Human AD Brains

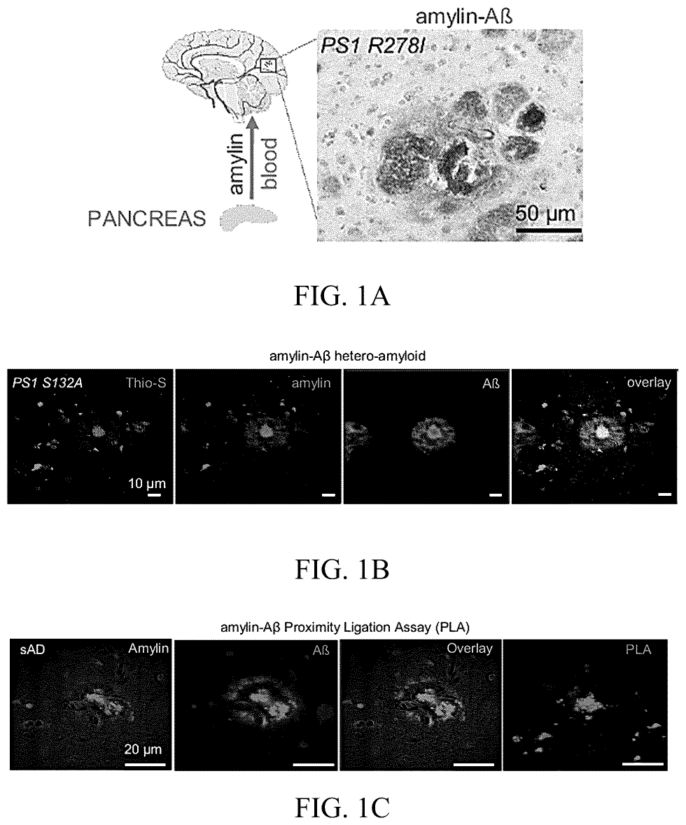

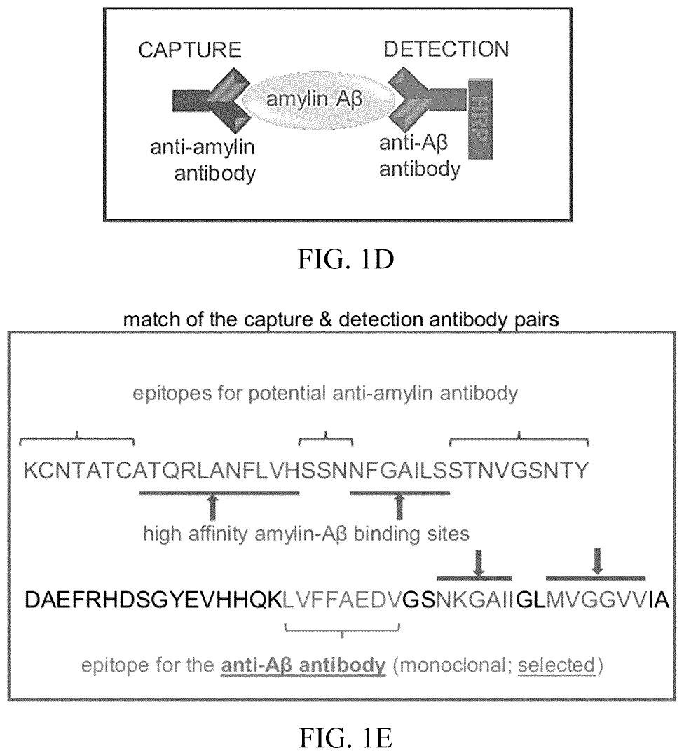

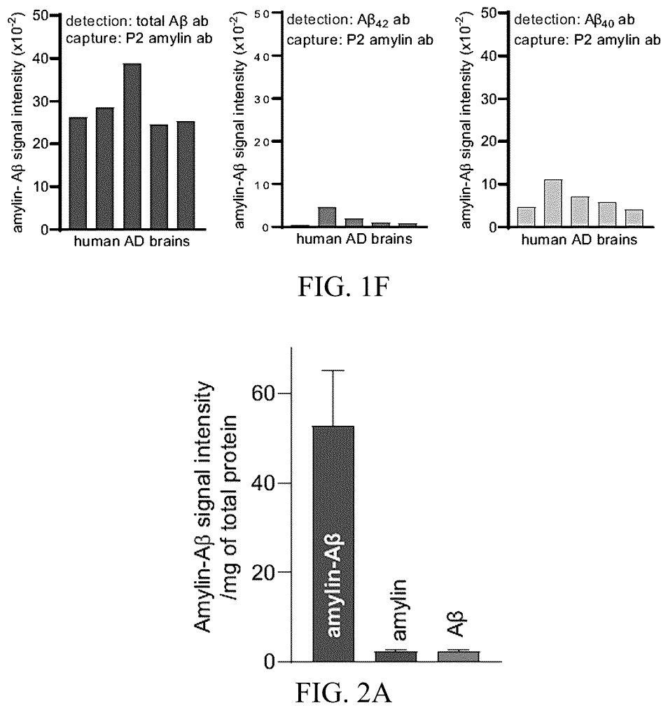

An example of cerebral amylin-Aβ co-deposition reported previously is shown in FIG. 1A. The section through the brain of a PS1 mutation carrier shows cerebral Aβ deposits (green) and amylin (brown) forming the plaque core and intercalated amylin-Aβ deposits. Amylin-Aβ co-deposits have biochemical characteristics of amyloid, as demonstrated by Thioflavin S (Thio-S) co-staining (FIG. 1B), and involve direct molecular amylin-Aβ binding, as suggested by the proximity ligation assay (PLA) (FIG. 1C). To assess molecular amylin-Aβ co-deposition in human AD brains quantitatively, we developed an amylin-Aβ sandwich ELISA that relies on an anti-Aβ antibody (detection) and an anti-amylin antibody (capture) (FIG. 1D).

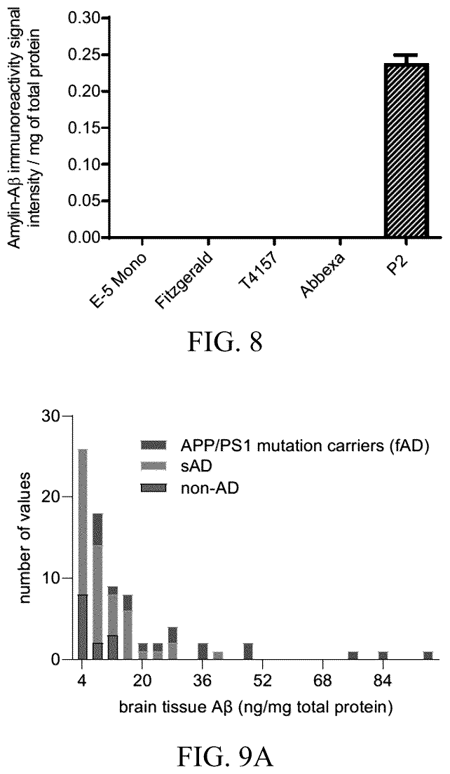

For co-aggregated amylin-Aβ (amylin-Aβ hetero-oligomers) to be detected by a sandwich ELISA, the molecular assembly must contain exposed epitopes that are distinct from amylin-Aβ binding sites on both amylin and Aβ peptides (FIG. 1E). These epitopes were identified based on analyses of amino acid sequences of amylin and Aβ peptides indicating regions on the amylin and Aβ peptides that promote amylin-Aβ oligomerization. For performing amylin-Aβ sandwich ELISA, three well-characterized monoclonal anti-Aβ antibodies were tested as potential detection antibodies, including those that recognize the 17-24 amino acids of Aβ (total Aβ antibody), the Aβ isoform ending at the 42^{nd} amino acid (Aβ₄₂ antibody) and the isoform ending at 40th amino acid (Aβ₄₀ antibody). For capturing antibodies, we tested both amylin C- and N-termini antibodies. Based on the immunoreactivity signal intensity measured in solutions of amylin-Aβ hetero-oligomers (FIG. 8), a polyclonal anti-amylin antibody (P2) designed to recognize a N-terminus amylin epitope was selected as the capture antibody. The P2 capture antibody matched with the anti-total Aβ detection antibody showed a robust amylin-Aβ immuno-reactivity signal intensity in human AD brain tissue homogenates (FIG. 1F). Therefore, our specific amylin-Aβ sandwich ELISA for measuring amylin-Aβ co-aggregation in AD brain tissue homogenates relies on a monoclonal anti-Aβ mid-domain antibody (detection) and a polyclonal anti-amylin antibody (capture). This assay will recognize only amylin-Aβ molecular assemblies that contain at least one exposed Aβ within amylin-positive molecular aggregates.

### Specificity of Sandwich ELISA to Detection of Prefibrillar Amylin-Aβ Hetero-Oligomers

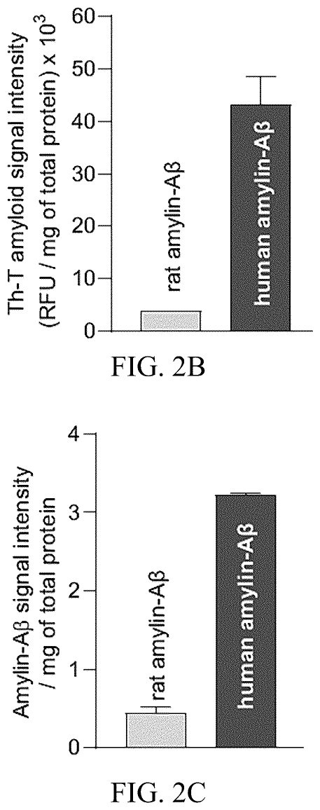

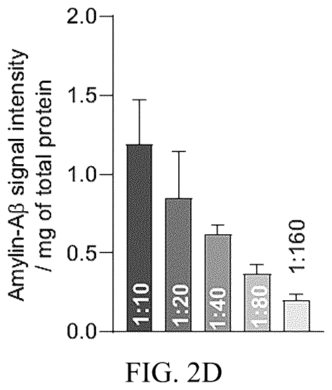

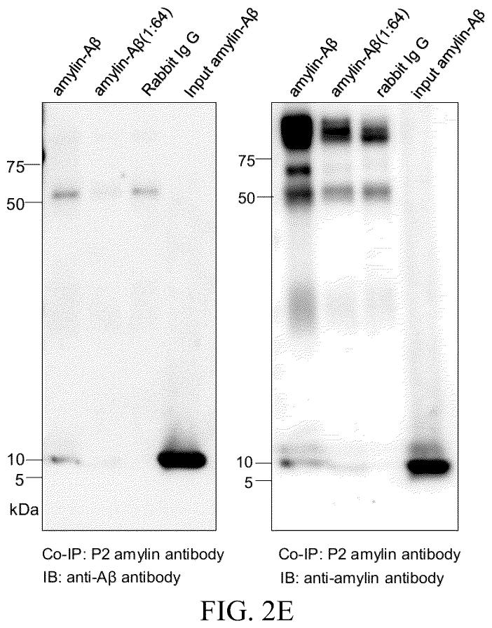

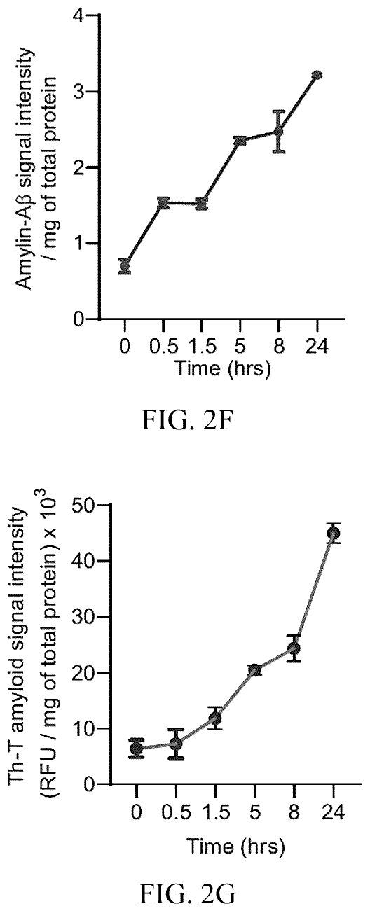

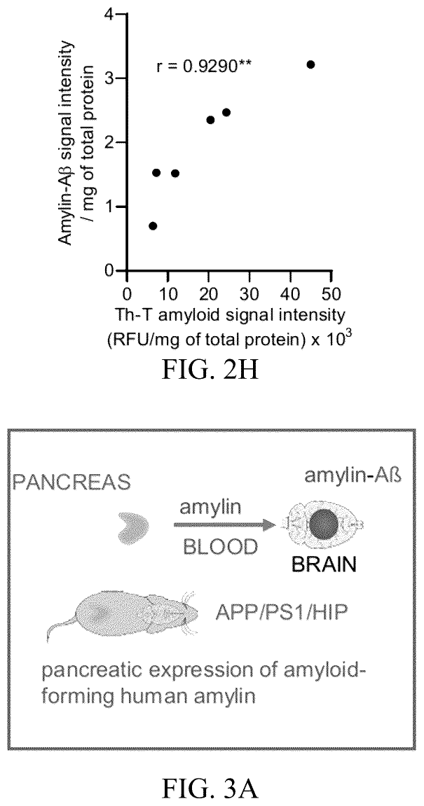

Specificity of amylin-Aβ sandwich ELISA was confirmed using a solution phase amylin-Aβ aggregation assay described in O'Nuallain et al. Synthetic human amylin and Aβ₄₀ peptides were incubated for promoting the amylin-Aβ aggregation and subjected to the amylin-Aβ sandwich ELISA. Protein solutions created by the incubation of similar concentrations of amylin or Aβ₄₀ alone, as used in preparing amylin-Aβ aggregates, provided negative controls for the detection of amylin-Aβ immunoreactivity. A robust amylin-Aβ immuno-reactivity signal was detected in co-aggregated amylin-Aβ solution, but not in the homogenous amylin and Aβ solutions (FIG. 2A). The aggregated amylin-Aβ solution was assayed for biochemical characteristics of amyloid by measuring the Thioflavin-T (Th-T) fluorescence signal intensity. Because rat amylin is non-amyloidogenic, negative controls for the amylin-Aβ amyloid characteristics were protein solutions created by incubation of equal concentrations of Aβ and rat amylin. Both Th-T fluorescence and amylin-Aβ ELISA signal intensities were greatly reduced in rat amylin-Aβ solutions compared to that in human amylin-Aβ solution (FIGS. 2B-2C). The average amylin-Aβ ELISA signal intensity in human amylin-Aβ co-aggregation assays is ˜7-fold higher than that corresponding to rat amylin-Aβ co-aggregation (FIG. 2C). The propensity of aggregate formation by rat amylin with Aβ is much lower than aggregate formation by human amylin with Aβ, consistent with previously published data.

From the same aliquot as in FIG. 2A, serial dilutions of amylin-Aβ hetero-oligomers were assayed using the amylin-Aβ sandwich ELISA (FIG. 2D) and the limit of detection (LOD) was estimated. The LOD of the amylin-Aβ sandwich ELISA is 0.02 ng/mg total protein. Next, we used the same anti-amylin and anti-Aβ antibodies as in the sandwich ELISA (i.e., P2 amylin antibody and HRP-conjugated anti-Aβ antibody) for co-immunoprecipitation of amylin in amylin-Aβ aggregates (undiluted and 1:64 diluted) followed by a direct Western blot analysis of Aβ in amylin IP eluates. Low molecular weight (˜10 kDa) hetero-oligomers (likely amylin-Aβ dimers) were present within the hetero-oligomeric solution (FIG. 2E). Following further dilution (1:64) of the input aliquot, low molecular weight (˜10 kDa) hetero-oligomers are faintly shown on Western blot, consistent with the lower sandwich ELISA signal intensity show in FIG. 2D.

To test whether prefibrillar amylin-Aβ oligomers can be detected via the sandwich ELISA method, the time evolution of the sandwich ELISA signal intensity in an incubated solution of amylin and Aβ (FIG. 2F) was analyzed in parallel for amyloid fibril formation via measurements of Th-T fluorescence (FIG. 2G). The measurements were conducted at 6 different time points (0, 0.5, 1.5, 5, 8 and 24-hours) post-incubation. The results show that increased Th-T fluorescence signal intensities are associated with greater sandwich ELISA signal intensities (r=0.93; P: 0.01) and a 3-fold increase from the baseline (FIG. 2H). It is interpreted that amylin-Aβ ELISA can detect prefibrillar amylin-Aβ hetero-oligomers.

### Sensitivity of Amylin-Aβ Sandwich ELISA to Dissociation of Amylin-Aβ Hetero-Oligomers

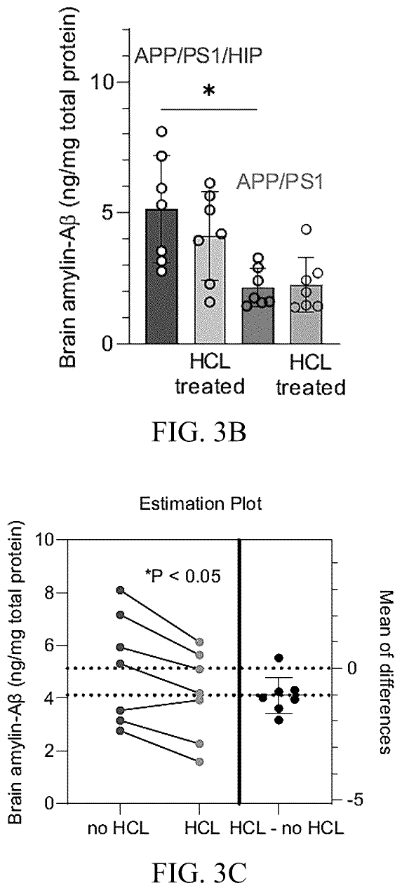

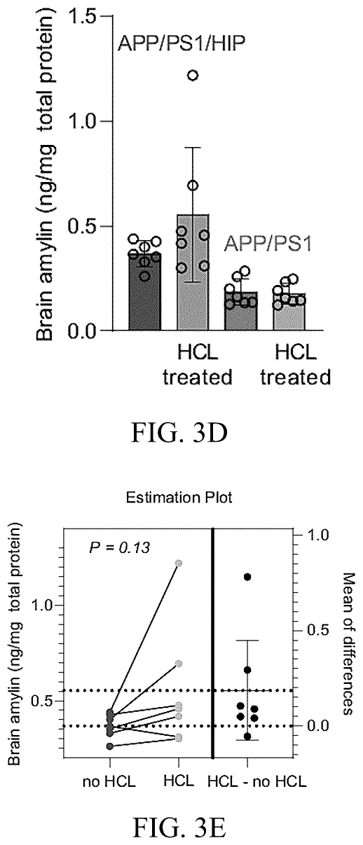

To further confirm that the amylin-Aβ sandwich ELISA is sensitive to amylin-Aβ hetero-oligomers formed in vivo, brain tissue homogenates from APP/PS1 rats expressing human amylin specifically in the pancreatic B-cells (i.e., APP/PS1/HIP rats) was assayed. APP/PS1 rats that express wild-type endogenous rat amylin were negative controls for the amyloidogenicity of human amylin. Full characterization of cerebral amylin-Aβ pathology and behavior deficits in APP/PS1/HIP versus APP/PS1 rats were recently published. Sandwich ELISA signal intensities were measured in APP/PS1/HIP and APP/PS1 brain tissue homogenates. The mean level of oligomerized amylin-Aβ is higher in APP/PS1/HIP brain tissue homogenates compared to those in the APP/PS1 rat group (Kruskal-Wallis one-way analysis of variance, P<0.01; FIG. 3B).

To assess the sensitivity of the ELISA to fragmentation of amylin-Aβ hetero-oligomers, brain tissue homogenates from same rats were treated with 1M hydrochloric acid (HCL) followed by the sandwich ELISA assay. Fragmenting of amylin-Aβ hetero-oligomers decreased sandwich ELISA signal intensities in APP/PS1/HIP rat brain tissue homogenates (FI. 3C). Although the sandwich ELISA detects rat amylin-Aβ hetero-oligomers in rat amylin-Aβ solutions (FIG. 2C), the average detection intensities corresponding to rat amylin-Aβ hetero-oligomers in HCl-treated versus untreated APP/PS1 brain tissue homogenates are similar (FIG. 3B). This may suggest the presence of low concentrations of rat amylin-Aβ hetero-oligomers in APP/PS1 rat brains. In the same APP/PS1/HIP rat brain homogenates as in FIG. 3B, fragmenting of amylin-Aβ hetero-oligomers appears associated with more solubilized amylin (FIG. 3D), although the analysis shows a large variability of the amylin ELISA signal intensity in HCl-treated versus non-treated tissue homogenates (FIG. 3E).

Taken together, the results in FIGS. 2 and 3 indicate that the amylin-Aβ sandwich ELISA measures the concentrations of co-aggregated amylin-Aβ (hetero-oligomers) in a linear fashion, and is sensitive to the Th-T biochemical characteristics of early stage amylin-Aβ hetero-oligomers and the dissociation of amylin-Aβ hetero-oligomers to monomers.

### Amylin-Aβ Hetero-Oligomer Levels in AD Versus Non-AD Brains

By using the amylin-Aβ sandwich ELISA, 76 human temporal cortex homogenates for amylin-Aβ hetero-oligomers were screened. Amylin and Aβ concentrations in brain tissue homogenates were measured using conventional ELISAs. As the positive control for cerebral mixed amylin-Aβ pathology, we used brain tissue homogenates from a subset of 18 APP and PS1 mutation carriers that have been previously described (the early-onset familial AD group; fAD). The assessment of clinical dementia and the neuropathologic features-neuritic amyloid plaques (Consortium to Establish a Registry for Alzheimer's Disease; CERAD) and Braak NFT stage in persons with sporadic AD (SAD) were previously described. The analyses included n=42 sAD brains that have Braak NFT stage I-VI and CERAD A, B or C, n=3 sAD brains that have CERAD none and NFT stage III-IV, and n=13 brains that have CERAD none and low Braak scores (0, I or II) (the non-AD group). Individuals were of similar age (sAD, 85.65±7.04 years versus non-AD, 87.22±7.30 years; P=0.482).

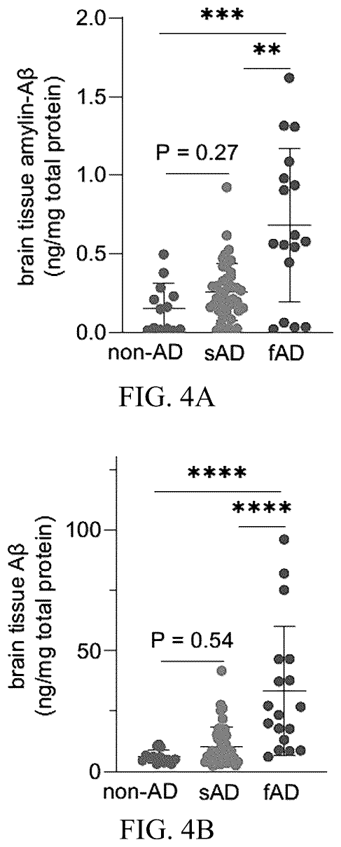

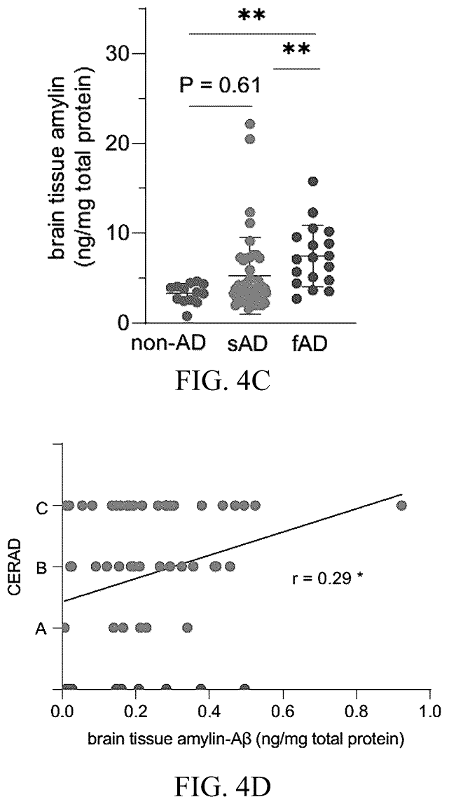

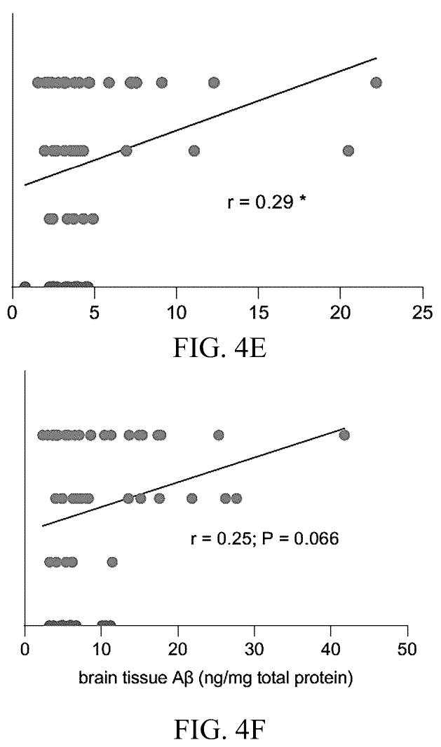

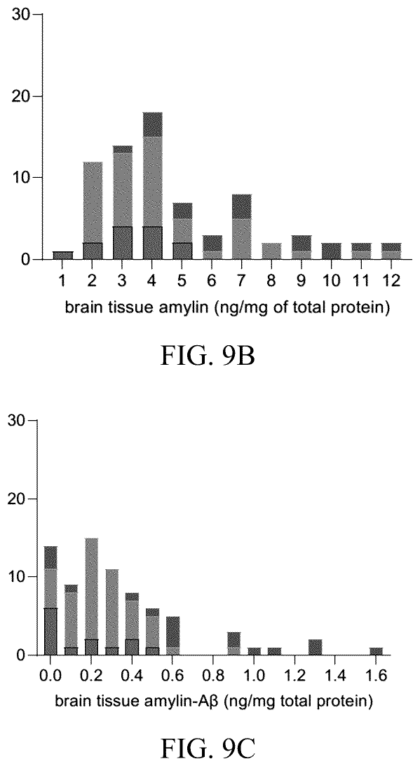

Frequency distributions of brain tissue amylin, Aβ and amylin-Aβ hetero-oligomer concentrations indicate distinct homeostasis of amylin and Aβ amyloidogenic peptides within the fAD and sAD groups (FIG. 9). Most of the disease brains had detectable amylin-Aβ hetero-oligomer concentrations, whereas about half of control subjects had brain tissue amylin-Aβ hetero-oligomers below our detection limit (0.02 ng/mg total protein) (n=6 samples with no ELISA evidence of brain amylin-Aβ hetero-oligomers; FIG. 9C). The mean level of oligomerized amylin-Aβ is higher in brains within the fAD group compared to those in the sAD and non-AD groups (one-way ANOVA, P<0.001 and P<0.01, respectively FIG. 4A), consistent with similar relationships between the average amylin and Aβ levels in fAD compared to sAD and non-AD groups (FIGS. 4B-4C). In the sAD group, the average Aβ and amylin levels are not significantly increased compared to those in the non-AD group (one-way ANOVA, P 0.05, FIGS. 4B-4C), which appears to explain why amylin-Aβ hetero-oligomer levels are only significantly increased in patients with fAD, but not sAD, relative to no-AD (FIG. 4A).

The potential relationship between amylin-Aβ hetero-oligomer levels and densities of amyloid plaques was next assessed. Brains that have CERAD none and low Braak scores (0, I or II) (green dots) were compared to brains that have CERAD A-C and Braak scores I-VI (red dots). Data from n=3 sAD brains that have CERAD none and higher Braak scores (III or IV) were not included in the initial analysis. The results show increased brain tissue amylin, Aβ and amylin-Aβ hetero-oligomer levels are more common in sAD brains compared to non-AD brains (FIGS. 4D-4F). The pairwise correlation coefficient suggests a possible relationship between brain tissue amylin-Aβ hetero-oligomer levels and frequent neuritic plaques (r=0.29; P<0.05) (FIG. 4D). This changed to r=0.23 (P<0.05) in the analysis that included sAD brains with CERAD none and high Braak scores.

Given the association between sAD pathology and advanced age, we assessed a potential relationship between cerebral amylin-Aβ hetero-oligomerization and age in sAD and non-AD groups. Overall, there is no correlation between cerebral amylin-Aβ hetero-oligomer accumulation and age (FIG. 5A). In sAD brains, however, there is an apparent propensity to lower levels of amylin-Aβ hetero-oligomer levels with increasing age (P=0.054), which may reflect the demonstrated shift of Aβ to plaque formation during age-related AD pathogenesis.

### Relationships Between Amylin-Aβ Hetero-Oligomerization and Brain Amylin and Aβ Levels in the Setting of Type-2 Diabetes Mellitus

Dysregulated amylin is a contributing factor to pathogenesis of both type-2 diabetes and AD. We conducted regression analyses of the relationships between amylin-Aβ hetero-oligomerization and brain amylin and Aβ levels (predictor variables) in subjects stratified on diagnosis of AD versus non-AD and diabetes versus non-diabetes (FIGS. 5-6). Age and sex were included as covariates in all analyses (see Methods for detailed regression models).

Our previous data showing a direct relationship between amylin and Aβ levels in AD brains suggest a possible effect modification in which the effect of brain amylin level on the amylin-Aβ hetero-oligomerization may depend on the Aβ level. To test this hypothesis, we employed a multiple linear regression model that includes brain amylin and Aβ levels as predictor variables, and a multiplicative (amylin×Aβ) interaction term. The interaction term is not associated with amylin-Aβ hetero-oligomerization (P=0.13) after including the main effect terms (brain amylin and Aβ levels). Brain amylin-Aβ hetero-oligomerization is associated with brain amylin levels only in the AD group (FIGS. 5B-5C). The regression coefficients are only marginally altered after adjusting for the brain Aβ levels (P=0.05); however, we retained the Aβ term in the multiple linear regression analysis. In the AD group, the regression equation is Y=0.1+0.03 amylin (P=0.045)+0.01 Aβ (P=0.050). For a given brain Aβ level, each ng/mg increase of brain amylin level is associated with an average increase in amylin-Aβ hetero-oligomers of 0.03 ng/mg. The intercept is 0.1 and has no biological meaning as brain amylin and Aβ levels are non-zero.

In groups stratified based on the diabetes status, the average level of oligomerized amylin-Aβ is higher in those with type-2 diabetes mellitus (diabetes group; n=28) versus those without (non-diabetes group; n=30) (FIG. 6A). The linear regression model shows amylin-Aβ hetero-oligomerization is associated with increased brain amylin level (P=0.006) after adjusting for the brain Aβ level (P=0.023) and the interaction term (amylin×Aβ) is significant (P=0.031), in the diabetes group. The regression equation is Y=0.048+0.041 amylin+0.012 Aβ-0.002 amylin×Aβ (FIG. 6B). For given brain Aβ level and magnitude of amylin×Aβ interaction, each ng/mg increase of brain amylin level is associated with an average increase in amylin-Aβ hetero-oligomers of 0.041 ng/mg. The intercept has no biological meaning, as brain amylin and Aβ levels are non-zero. The interaction coefficient is negative suggesting a reduction of effect modification of brain amylin level on the amylin-Aβ hetero-oligomerization at higher brain Aβ levels (i.e., a possible saturation effect).

To assess the extent of amylin-Aβ hetero-oligomerization effect on the link between AD and type-2 diabetes, we used logistic regression models based on brain amylin, Aβ and amylin-Aβ hetero-oligomer levels as covariates. We then compared the goodness-of-fit of the model in persons with AD and diabetes versus persons with AD without diabetes. A model based on brain amylin, amylin-Aβ and age as covariates has a >15% increase of the area under the Receiver Operating Characteristic (ROC) curve (pink curve, ROC area=0.875; FIG. 6C) compared to the ROC of a logistic model based on brain Aβ level and age as predictor variables (gray curve, ROC area=0.698; FIG. 6C). Consistent with this result, our data show improved confidence interval (CI: 1.0-1.2) for the odds ratio (OR=1.1; P=0.031) corresponding to the amylin-Aβ-based model, compared to the CI (0.98-1.3) for the OR=1.14 (P=0.088) corresponding to the Aβ-based model. Therefore, the amylin-Aβ-based model improves the prediction of AD/non-AD status, compared to a logistic model based on the Aβ-based model, in the setting of type-2 diabetes.

In contrast to the diabetes group, in which the regression coefficients of the linear regression model are significant (FIG. 6B), the relationship between brain amylin-Aβ hetero-oligomerization and brain amylin levels adjusted for the brain Aβ levels is not significant (P<0.05) (FIG. 6D), in persons without diabetes. Furthermore, ROC areas corresponding to the two logistic regression models (i.e., amylin-Aβ versus Aβ-based models) are similar (i.e., 0.751 versus 0.714; FIG. 6E), in the non-diabetes AD group. Either brain amylin-Aβ or Aβ-based models similarly predict AD/non-AD status in persons without diabetes.

### Circulating Amylin-Aβ Hetero-Oligomers in Blood

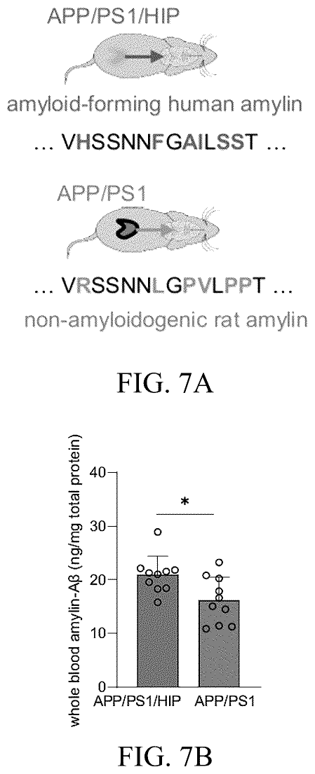

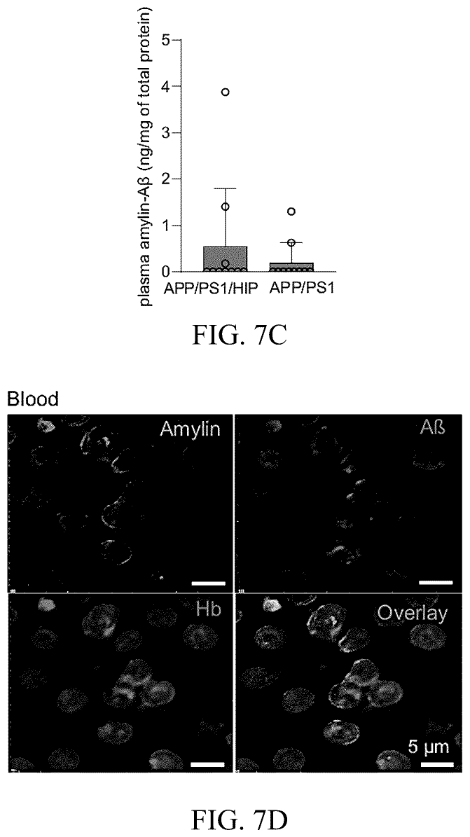

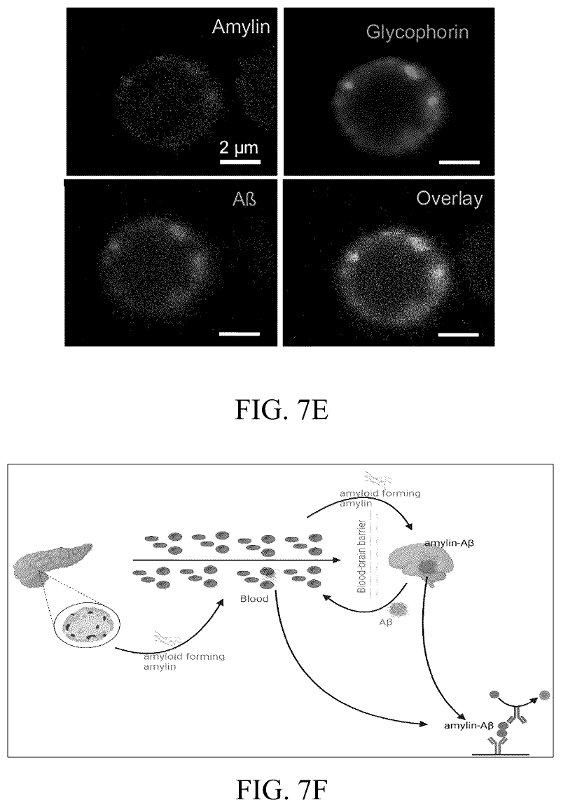

Using the amylin-Aβ sandwich ELISA, amylin-Aβ oligomer levels in whole blood lysates from APP/PS1 rats expressing amyloid forming amylin in the pancreas (male APP/PS1/HIP rats; age, 16-months) was measured. Whole blood lysates from male APP/PS1 littermates were the negative controls for amyloidogenicity of human amylin. Our results show increased levels of amylin-Aβ hetero-oligomers in whole blood lysates obtained from APP/PS1/HIP rats compared to those from APP/PS1 littermates (FIG. 7A). Plasma amylin-Aβ ELISA indicate amylin-Aβ hetero-oligomers were below the detection limit of our ELISA in most samples (FIG. 7B). FIG. 7D shows representative confocal microscopic images of red blood cells (RBCs) from APP/PS1/HIP rats; samples were triple-stained for amylin, Aβ and hemoglobin, a protein that is specific for RBCs. To further assess the potential amylin-Aβ co-localization on the RBC membrane, RBC samples were triple-stained for amylin, Aβ and glycophorin (FIG. 7E), a protein that is expressed within the RBC membrane. Taken together, the results suggest amylin-Aβ co-deposits on circulating RBCs. Circulating amylin-Aβ hetero-oligomers and those accumulating within brain tissues have shared prefibrillar molecular structures and can be detected by the amylin-Aβ ELISA (FIG. 7E).

### Discussion

Accumulating data from studies in humans suggest an association between pancreatic amyloid-forming amylin and cerebral Aβ pathology. The results indicate cerebral amylin-Aβ plaques form through mechanisms that involve amylin-Aβ co-aggregation at the molecular level. As reflected above, amylin-Aβ hetero-oligomers can be measured by an amylin-Aβ sandwich ELISA. The utility of this assay is demonstrated by analysis of post-mortem brain tissues collected from cognitively unaffected individuals and persons with AD. Using transgenic AD-model rats, we show that this new assay can detect circulating amylin-Aβ hetero-oligomers in the blood and is sensitive to the dissociation of amylin-Aβ hetero-oligomers to monomers.

The sensitivity of the sandwich ELISA to detect amylin-Aβ hetero-oligomers depends on matching of the capture and detection antibody pairs to recognize exposed epitopes that are distinct from amylin-Aβ binding sites on both amylin and Aβ peptides. The antibody against total Aβ recognizes an epitope distinct from the high affinity amylin-Aβ binding sites, which facilitates the increased sensitivity of the sandwich amylin-Aβ ELISA assay when carried out using anti-total Aβ antibody as the detection antibody. In contrast, the antibodies against the Aβ isoform ending at the 42^{nd} amino acid (Aβ₄₂ antibody) and the isoform ending at 40th amino acid (Aβ₄₀ antibody) recognize epitopes that appear involved in amylin-Aβ co-aggregation. The Aβ₄₂ and Aβ₄₀ antibodies appear less sensitive to amylin-Aβ hetero-oligomer detection. Based on the results and previous analyses of amylin-Aβ oligomerization ex vivo, it is concluded that the N-terminal epitope of amylin may not be involved in the amylin-Aβ aggregate formation. Related to this point, human amylin differs from rodent amylin by 6 amino acids at the C-terminus. Because of the similarity between human and rat amylin, the polyclonal P2 N-terminus amylin antibody cross-reacts with rat amylin, which may explain the presence of a weak Th-T fluorescence signal intensity detected in the rat amylin-Aβ aggregates. Consistent with this result and previously published data, amylin-Aβ ELISA signal intensities in human amylin-Aβ co-aggregation assays is about 7-fold higher than that corresponding to rat amylin-Aβ co-aggregation.

The evolution of the sandwich ELISA signal in an incubated solution of amylin and Aβ peptides analyzed in parallel for amyloid fibril formation via measurements of Th-T fluorescence indicates that the sandwich ELISA may detect prefibrillar amylin-Aβ hetero-oligomers. In APP/PS1/HIP rats, circulating amylin-Aβ hetero-oligomers and those accumulating within brain tissues appear to share prefibrillar molecular structures that can be detected by the amylin-Aβ ELISA. Both human amylin and Aβ have high propensity to attach to cellular membranes, which is shown by amylin-Aβ co-deposition on circulating RBCs. Amylin deposition on RBCs is common in persons with diabetes-related microvascular complications. It is hypothesized that amylin-Aβ co-deposits on circulating RBCs in APP/PS1/HIP rats reflect amylin-Aβ hetero-oligomerization within the microvasculature. In the rodent model of APP/PS1 dementia, the reported level of Aβ in the blood is in the ˜10-70 ng/ml range.

Previous studies showed that amylin concentrations in blood and cerebrospinal fluid are higher in AD than in cognitively unaffected persons suggesting a possible relationship between circulating amylin levels and the propensity of amylin to accumulate in the brain. Because of the accumulating evidence suggesting that pancreatic amyloid-forming amylin synergistically co-aggregates with vascular and parenchymal Aβ, it is important to be able to specifically measure molecular amylin-Aβ species in a range of biological samples. A lack of rapid, accessible, scalable and accurate biochemical methods to quantify amylin-Aβ interaction prevents larger sample studies, hindering the development of therapeutic strategies to prevent brain amylin accumulation and interaction with AD pathology. The results confirm that amylin-Aβ hetero-oligomers can be detected and quantified, and may serve as a marker of amylin-Aβ established interaction and its correlation with AD pathology.

In groups stratified based on the diabetes status, the average level of amylin-Aβ hetero-oligomerization is higher in those with type-2 diabetes mellitus. The slopes with respect to brain amylin levels appear different between the groups, suggesting that, in a larger cohort that includes individuals without diabetes, there may occur effect modification and the measures of association in the subgroups may differ from one another. We found a significant relationship between brain amylin-Aβ hetero-oligomerization and brain amylin levels adjusted for the brain Aβ levels, in the diabetes group. Potential mechanisms accounting for diabetes-associated increased amylin-Aβ hetero-oligomerization may involve hypersecretion of amyloid-forming pancreatic amylin. The interaction coefficient is negative suggesting a reduction of effect modification of brain amylin level on the amylin-Aβ hetero-oligomerization at higher brain Aβ levels (i.e., saturation effect). In the diabetes group, the logistic regression model based on brain amylin and amylin-Aβ hetero-oligomer levels as covariates improves the prediction of AD/non-AD status, compared to a logistic model based on brain Aβ level and age as predictor variables. Taken together, the results suggest that it may be important to account for brain tissue amylin-Aβ hetero-oligomerization in assessing the effect of diabetes on AD.

### REFERENCES

All publications, patents, and patent applications mentioned in this specification are herein incorporated by reference to the same extent as if each individual publication, patent, or patent application was specifically and individually indicated to be incorporated by reference, including the references set forth in the following list:
1. Kellar, D., and Craft, S. (2020) Brain insulin resistance in Alzheimer's disease and related disorders: mechanisms and therapeutic approaches. Lancet Neurol. 19, 758-766
2. Biessels, G. J., and Despa, F. (2018) Cognitive decline and dementia in diabetes mellitus: mechanisms and clinical implications. Nat Rev Endocrinol. 14, 591-604
3. Kahn, S. E., D'Alessio, D. A., Schwartz, M. W., Fujimoto, W. Y., Ensinck, J. W., Taborsky, G. J., et al. (1990) Evidence of cosecretion of islet amyloid polypeptide and insulin by beta-cells. Diabetes. 39, 634-638
4. Banks, W. A., and Kastin, A. J. (1998) Differential permeability of the blood-brain barrier to two pancreatic peptides: insulin and amylin. Peptides (N.Y.). 19, 883-889
5. Hay, D. L., Chen, S., Lutz, T. A., Parkes, D. G., and Roth, J. D. (2015) Amylin: pharmacology, physiology, and clinical potential. Pharmacol Rev. 67, 564-600
6. Westermark, P., Andersson, A., and Westermark, G. T. (2011) Islet amyloid polypeptide, islet amyloid, and diabetes mellitus. Physiol Rev. 91, 795-826
7. Jurgens, C. A., Toukatly, M. N., Fligner, C. L., Udayasankar, J., Subramanian, S. L., Zraika, S., et al. (2011) β-Cell loss and β-Cell apoptosis in human type-2 diabetes are related to islet amyloid deposition. Am J Pathol. 178, 2632
8. Hoppener, J. W. M., Ahren, B., and Lips, C. J. M. (2000) Islet amyloid and type-2 diabetes mellitus. N Engl J Med. 343, 411-419
9. Jackson, K., Barisone, G. A., Diaz, E., Jin, L. W., DeCarli, C., and Despa, F. (2013) Amylin deposition in the brain: A second amyloid in Alzheimer disease? Ann Neurol. 74, 517-526
10. Oskarsson, M. E., Paulsson, J. F., Schultz, S. W., Ingelsson, M., Westermark, P., and Westermark, G. T. (2015) In vivo seeding and cross-seeding of localized amyloidosis. Am J Pathol. 185, 834-846
11. Martinez-Valbuena, I., Valenti-Azcarate, R., Amat-Villegas, I., Riverol, M., Marcilla, I., de Andrea, C. E., et al. (2019) Amylin as a potential link between type-2 diabetes and alzheimer disease. Ann Neurol. 86, 539-551
12. Ly, H., Verma, N., Sharma, S., Kotiya, D., Despa, S., Abner, E. L., et al. (2021) The association of circulating amylin with β-amyloid in familial Alzheimer's disease. Alzheimer's & Dementia: Translational Research & Clinical Interventions. 7, 20-21
13. Verma, N., Velmurugan, G. V., Winford, E., Coburn, H., Kotiya, D., Leibold, N., et al. (2023) Aβ efflux impairment and inflammation linked to cerebrovascular accumulation of amyloid-forming amylin secreted from pancreas. Commun Biol. 10.1038/S42003-022-04398-2
14. O'Nuallain, B., Williams, A. D., Westermark, P., and Wetzel, R. (2004) Seeding specificity in amyloid growth induced by heterologous fibrils. J Biol Chem. 279, 17490-17499
15. Yan, L. M., Velkova, A., Tatarek-Nossol, M., Andreetto, E., and Kapurniotu, A. (2007) IAPP mimic blocks Abeta cytotoxic self-assembly: cross-suppression of amyloid toxicity of Abeta and IAPP suggests a molecular link between Alzheimer's disease and type II diabetes. Angew Chem Int Ed Engl. 46, 1246-1252
16. Andreetto, E., Yan, L. M., Tatarek-Nossol, M., Velkova, A., Frank, R., and Kapurniotu, A. (2010) Identification of hot regions of the Abeta-IAPP interaction interface as high-affinity binding sites in both cross- and self-association. Angew Chem Int Ed Engl. 49, 3081-3085
17. Yan, L.-M., Velkova, A., and Kapurniotu, A. (2014) Molecular characterization of the hetero-assembly of β-amyloid peptide with islet amyloid polypeptide. Curr Pharm Des. 20, 1182-1191
18. Kapurniotu, A. (2020) Enlightening amyloid fibrils linked to type-2 diabetes and cross-interactions with Aβ. Nat Struct Mol Biol. 27, 1006-1008
19. Taş, K., Volta, B. D., Lindner, C., el Bounkari, O., Hille, K., Tian, Y., et al. (2022) Designed peptides as nanomolar cross-amyloid inhibitors acting via supramolecular nanofiber co-assembly. Nature Communications 2022 13:1. 13, 1-22
20. Bharadwaj, P., Solomon, T., Sahoo, B. R., Ignasiak, K., Gaskin, S., Rowles, J., et al. (2020) Amylin and beta amyloid proteins interact to form amorphous heterocomplexes with enhanced toxicity in neuronal cells. Scientific Reports 2020 10:1. 10, 1-14
21. Wang, Y., and Westermark, G. T. (2021) The Amyloid Forming Peptides Islet Amyloid Polypeptide and Amyloid β Interact at the Molecular Level. Int J Mol Sci. 10.3390/IJMS222011153
22. Forny-Germano, L., Lyra E Silva, N. M., Batista, A. F., Brito-Moreira, J., Gralle, M., Boehnke, S. E., et al. (2014) Alzheimer's disease-like pathology induced by amyloid-β oligomers in nonhuman primates. J Neurosci. 34, 13629-13643
23. Savage, M. J., Kalinina, J., Wolfe, A., Tugusheva, K., Korn, R., Cash-Mason, T., et al. (2014) A Sensitive Aβ Oligomer Assay Discriminates Alzheimer's and Aged Control Cerebrospinal Fluid. Journal of Neuroscience. 34, 2884-2897
24. Watanabe, H., Xia, D., Kanekiyo, T., Kelleher, R. J., and Shen, J. (2012) Familial frontotemporal dementia-associated presenilin-1 c.548G>T mutation causes decreased mRNA expression and reduced presenilin function in knock-in mice. J Neurosci. 32, 5085-5096
25. Westermark, P., Engstrom, U., Johnson, K. H., Westermark, G. T., and Betsholtz, C. (1990) Islet amyloid polypeptide: pinpointing amino acid residues linked to amyloid fibril formation. Proc Natl Acad Sci USA. 87, 5036
26. Nelson, P. T., Pious, N. M., Jicha, G. A., Wilcock, D. M., Fardo, D. W., Estus, S., et al. (2013) APOE-82 and APOE-84 Correlate with Increased Amyloid Accumulation in Cerebral Vasculature. J Neuropathol Exp Neurol. 72, 708
27. A. Schmitt, F., T. Nelson, P., Abner, E., Scheff, S., A. Jicha, G., Smith, C., et al. (2012) University of Kentucky Sanders-Brown healthy brain aging volunteers: donor characteristics, procedures and neuropathology. Curr Alzheimer Res. 9, 724-733
28. Nelson, P. T., Jicha, G. A., Schmitt, F. A., Liu, H., Davis, D. G., Mendiondo, M. S., et al. (2007) Clinicopathologic correlations in a large Alzheimer disease center autopsy cohort: neuritic plaques and neurofibrillary tangles “do count” when staging disease severity. J Neuropathol Exp Neurol. 66, 1136-1146
29. Braak, H., and Braak, E. (2004) Neuropathological stageing of Alzheimer-related changes. Acta Neuropathol. 82, 239-259
30. Mirra, S. S., Heyman, A., McKeel, D., Sumi, S. M., Crain, B. J., Brownlee, L. M., et al. (1991) The Consortium to Establish a Registry for Alzheimer's Disease (CERAD). Part II. Standardization of the neuropathologic assessment of Alzheimer's disease. Neurology. 41, 479-486
31. Jack, C. R., Bennett, D. A., Blennow, K., Carrillo, M. C., Dunn, B., Haeberlein, S. B., et al. (2018) NIA-AA Research Framework: Toward a biological definition of Alzheimer's disease. Alzheimers Dement. 14, 535-562
32. Wang, J., Dickson, D. W., Trojanowski, J. Q., and Lee, V. M. Y. (1999) The levels of soluble versus insoluble brain Abeta distinguish Alzheimer's disease from normal and pathologic aging. Exp Neurol. 158, 328-337
33. Aguzzi, A., and Haass, C. (2003) Games played by rogue proteins in prion disorders and Alzheimer's disease. Science. 302, 814-818
34. Janson, J., Ashley, R. H., Harrison, D., McIntyre, S., and Butler, P. C. (1999) The mechanism of islet amyloid polypeptide toxicity is membrane disruption by intermediate-sized toxic amyloid particles. Diabetes. 48, 491-498
35. Haass, C., and Selkoe, D. J. (2007) Soluble protein oligomers in neurodegeneration: lessons from the Alzheimer's amyloid beta-peptide. Nat Rev Mol Cell Biol. 8, 101-112
36. Verma, N., Liu, M., Ly, H., Loria, A., Campbell, K. S., Bush, H., et al. (2020) Diabetic microcirculatory disturbances and pathologic erythropoiesis are provoked by deposition of amyloid-forming amylin in red blood cells and capillaries. Kidney Int. 97, 143-155
37. Zhang, L., Yang, C., Li, Y., Niu, S., Liang, X., Zhang, Z., et al. (2021) Dynamic Changes in the Levels of Amyloid-β42 Species in the Brain and Periphery of APP/PS1 Mice and Their Significance for Alzheimer's Disease. Front Mol Neurosci. 14, 183
38. Zhu, H., Tao, Q., Ang, T. F. A., Massaro, J., Gan, Q., Salim, S., et al. (2019) Association of plasma amylin concentration with Alzheimer disease and brain structure in older adults. JAMA Netw Open. 2, e199826-e199826
39. Royall, D. R., and Palmer, R. F. (2019) Blood-based protein mediators of senility with replications across biofluids and cohorts. Brain Commun. 10.1093/BRAINCOMMS/FCZ036
40. Saradhi, M., Krishna, B., Mukhopadhyay, G., and Tyagi, R. K. (2005) Purification of full-length human pregnane and xenobiotic receptor: polyclonal antibody preparation for immunological characterization. Cell Res. 15, 785-95
41. Armbruster, D. A., and Pry, T. (2008) Limit of Blank, Limit of Detection and Limit of Quantifyion. Clin Biochem Rev. 29, S49
42. Kohl, T. O., and Ascoli, C. A. (2017) Indirect Immunometric ELISA. Cold Spring Harb Protoc. 2017, 396-401
43. Ly, H., Verma, N., Wu, F., Liu, M., Saatman, K. E., Nelson, P. T., et al. (2017) Brain microvascular injury and white matter disease provoked by diabetes-associated hyperamylinemia. Ann Neurol. 82, 208-222
44. Verma, N., Ly, H., Liu, M., Chen, J., Zhu, H., Chow, M., et al. (2016) Intraneuronal amylin deposition, peroxidative membrane injury and increased IL-1β synthesis in rains of Alzheimer's disease patients with Type-2 diabetes and in diabetic HIP rats. J Alzheimers Dis. 53, 259-272
45. Kotiya, et al. (2023) Rapid, scalable assay of amylin-β amyloid co-aggregation in brain tissue and blood. J. Biol Chem. Vol. 299, Iss. 5 104682.

It will be understood that various details of the presently disclosed subject matter can be changed without departing from the scope of the subject matter disclosed herein. Furthermore, the foregoing description is for the purpose of illustration only, and not for the purpose of limitation.

What is claimed is:

1. A method to detect or quantify amylin-beta amyloid (Aβ) aggregate, comprising:
coating a solid surface with a capture antibody that specifically binds to one or more epitopes of amylin-Aβ aggregate;
contacting the coated solid surface with a biological sample to form a complex between the capture antibody and amylin-Aβ aggregate from the biological sample;
contacting the complex between the capture antibody and the amylin-Aβ aggregate from the biological sample with a detection antibody, the detection antibody having a detection enzyme conjugated thereto and specifically binding to an epitope of amylin-Aβ aggregate to form a complex between the capture antibody, the amylin-Aβ aggregate from the biological sample, and the detection antibody;
contacting the detection enzyme with a substrate; and
at least one of detecting the amylin-Aβ aggregate from the biological sample using a signal produced by a reaction between the substrate and the detection enzyme and quantifying the amylin-Aβ aggregate from the biological sample using the signal produced by a reaction between the substrate and the detection enzyme.

2. The method of claim 1, wherein the capture antibody is an anti-amylin antibody, and the detection antibody is an anti-Aβ antibody.

3. The method of claim 2, wherein the one or more epitopes that the anti-amylin antibody specifically binds to includes an exposed epitope of an amylin peptide of amylin-Aβ aggregate, and wherein the epitope of the amylin-Aβ aggregate that the anti-Aβ antibody specifically binds to is an exposed epitope of an Aβ peptide of amylin-Aβ aggregate.

4. The method of claim 2, wherein the capture antibody is a polyclonal antibody, and wherein the detection antibody is a monoclonal antibody.

5. The method of claim 4, wherein the one or more epitopes that the anti-amylin antibody specifically binds to includes at least two epitopes, with each epitope of the at least two epitopes comprising a sequence selected from SEQ ID NO: 3, SEQ ID NO: 4, and SEQ ID NO: 5.

6. The method of claim 4, wherein the one or more epitopes that the anti-amylin antibody specifically binds to includes a first epitope comprising SEQ ID NO: 3, a second epitope comprising SEQ ID NO: 4, and a third epitope comprising SEQ ID NO: 5.

7. The method of claim 6, wherein the first epitope, the second epitope, and the third epitope are each an exposed epitope of an amylin peptide of amylin-Aβ aggregate.

8. The method of claim 2, wherein the capture antibody includes one or more paratopes, with each paratope of the one or more paratopes comprising a sequence which is at least 90% homologous to the amino acid sequence of SEQ ID NO: 7.

9. The method of claim 2, wherein the detection antibody comprises a sequence selected from SEQ ID NO: 8, SEQ ID NO: 9, and SEQ ID NO: 10.

10. The method of claim 2, wherein the epitope that the detection antibody specifically binds to comprises SEQ ID NO: 6.

11. The method of claim 1, wherein the limit of detection for the amylin-Aβ aggregate from the biological sample is 0.02 ng/mg total protein.

12. The method of claim 1, wherein the biological sample is a blood sample.

13. The method of claim 1, wherein the biological sample is brain tissue.

14. The method of claim 1, wherein the biological sample is obtained from a subject suffering from at least one of type-2 diabetes and Alzheimer's disease.

15. The method of claim 1, wherein the amylin-Aβ aggregate from the biological sample is prefibrillar amylin-Aβ aggregate.

16. A kit, comprising:
an anti-amylin antibody that specifically binds to one or more epitopes of amylin-Aβ aggregate; and
an anti-Aβ antibody that specifically binds to an epitope of the amylin-Aβ aggregate that is distinct from the one or more epitopes of the amylin-Aβ aggregate that the anti-amylin antibody specifically binds to.

17. The kit of claim 16, wherein the one or more epitopes that the anti-amylin antibody specifically binds to includes an epitope comprising a sequence selected from SEQ ID NO: 3, SEQ ID NO: 4, and SEQ ID NO: 5.

18. The kit of claim 16, wherein the anti-amylin antibody is a polyclonal antibody, and wherein the one or more epitopes of the amylin-Aβ aggregate that the anti-amylin antibody specifically binds to includes at least two epitopes, with each epitope of the at least two epitopes comprising a sequence selected from SEQ ID NO: 3, SEQ ID NO: 4, and SEQ ID NO: 5.

19. The kit of claim 16, wherein the one or more epitopes of the amylin-Aβ aggregate that the anti-amylin antibody specifically binds to includes a first epitope comprising SEQ ID NO: 3, a second epitope comprising SEQ ID NO: 4, and a third epitope comprising SEQ ID NO: 5.

20. The kit of claim 19, wherein the first epitope, the second epitope, and the third epitope are each an exposed epitope of an amylin peptide of the amylin-Aβ aggregate.

21. The kit of claim 16, wherein the anti-amylin antibody comprises one or more paratopes, with each paratope of the one or more paratopes comprising a sequence which is at least 90% homologous to the amino acid sequence of SEQ ID NO: 7.

22. The kit of claim 16, wherein the anti-Aβ antibody is a monoclonal antibody.

23. The kit of claim 16, wherein the anti-Aβ antibody comprises a sequence selected from SEQ ID NO: 8, SEQ ID NO: 9, and SEQ ID NO: 10.

24. The kit of claim 16, wherein the epitope that the anti-Aβ antibody specifically binds to comprises SEQ ID NO: 6.

25. The kit of claim 16, and further comprising:
a detection enzyme linked to the anti-Aβ antibody.

26. The kit of claim 25, and further comprising at least one of:
a microplate including a well for receiving the anti-amylin antibody and the anti-Aβ antibody;
a washing buffer;
a substrate for reaction with the detection enzyme; and
a stop solution for terminating a reaction between the substrate and the detection enzyme.

27. A kit, comprising:
a polyclonal anti-amylin antibody that specifically binds to two or more epitopes of the amylin-Aβ aggregate, with each epitope of the two or more epitopes comprising a sequence selected from SEQ ID NO: 3, SEQ ID NO: 4, and SEQ ID NO: 5; and
a monoclonal anti-Aβ antibody that specifically binds to an epitope of the amylin-Aβ aggregate comprising SEQ ID NO: 6.

28. The kit of claim 27, wherein the anti-amylin antibody includes multiple paratopes, with each paratope of the multiple paratopes comprising a sequence which is at least 90% homologous to the amino acid sequence of SEQ ID NO: 7, and wherein a paratope of the anti-Aβ antibody comprises SEQ ID NO: 8.


SEQUENCE LISTING (10 sequences)

SEQ ID NO: 1 - protein, 37 aa, Synthetic Construct [sequence omitted]

SEQ ID NO: 2 - protein, 42 aa, Synthetic Construct [sequence omitted]

SEQ ID NO: 3 - protein, 7 aa, Synthetic Construct
KCNTATC

SEQ ID NO: 4 - skipped

SEQ ID NO: 5 - protein, 9 aa, Synthetic Construct
STNVGSNTY

SEQ ID NO: 6 - protein, 7 aa, Synthetic Construct
VFFAEDV

SEQ ID NO: 7 - protein, 21 aa, Oryctolagus cuniculus
CKCNTATCATQRLANFLVHSS

SEQ ID NO: 8 - protein, 5 aa, Mus musculus
VFFAE

SEQ ID NO: 9 - protein, 7 aa, Mus musculus
VGGVVIA

SEQ ID NO: 10 - protein, 5 aa, Mus musculus
VGGVV